(19) United States
(12) Patent Application Publication
(10) Pub. No.: US 2022/0098155 A1
(43) Pub. Date: Mar. 31, 2022

(54) NOVEL PATHWAY FOR THE SYNTHESIS OF DIAZIRINES, THAT MAY OR MAY NOT BE ENRICHED IN NITROGEN-15

(71) Applicants: UNIVERSITE DE ROUEN NORMANDIE, MONT SAINT AIGNAN, (FR); UNIVERSITE DE CAEN NORMANDIE, CAEN (FR); INSTITUT NATIONAL DES SCIENCES APPLIQUEES DE ROUEN (INSA), SAINT ETIENNE DU ROUVRAY (FR); CENTRE NATIONAL DE LA RECHERCHE SCIENTIFIQUE (CNRS), PARIS (FR); ECOLE NATIONALE SUPERIEURE D'INGENIEURS DE CAEN, CAEN (FR)

(72) Inventors: Vincent REBOUL, DOUVRES LA DELIVRANDE (FR); Xavier FRANCK, BOIS GUILLAUME (FR); Thomas GLACHET, CAEN (FR); Hamid MARZAG, ORLEANS (FR)

(21) Appl. No.: 17/292,832

(22) PCT Filed: Nov. 14, 2019
(86) PCT No.: PCT/EP2019/081393

§ 371 Date: Nov. 12, 2021

(30) Foreign Application Priority Data
Nov. 14, 2018 (FR) 1860507

(51) Int. Cl. C07D 229/02

(52) U.S. Cl. CPC C07D 229/02

(57) ABSTRACT

The present invention concerns a novel method for synthesising diazirines, that may or may not be enriched in nitro-gen-15, from amino acids or imines, via a one-pot synthesis method, comprising the reaction of the starting amino acid or imine with ammonia, which may or may not be enriched in nitrogen-15, and a hypervalent iodine oxidant. The present invention also relates to a method for synthesising ammonia enriched in nitrogen-15. The invention also concerns certain diazirines of formula (I) likely to be obtained by the claimed synthesis method, and also refers to the ¹⁵N₂-diazirines of formula (I′). The claimed diazirines can be used in photoaffinity labelling. The ¹⁵N₂-diazirines can also be used in hyperpolarisation, in particular in the medical imaging field.

The present invention concerns a novel method for synthesizing diazirines, eventually enriched in nitrogen-15, from amino acids or imines, via a one-pot synthesis method. The present invention also relates to a method for the preparation of an ammonia solution enriched in ¹⁵NH₃ and new diazirine compounds that are or are not enriched in nitrogen-15. These diazirines can be used in photoaffinity labelling. ¹⁵N₂-diazirines can also be used in hyperpolarization.

Diazirines are three-membered heterocyclic compounds with a nitrogen-nitrogen double bond and a sp³ carbon. They are generally obtained in several steps from carbonyl compounds. These carbonyl compounds are transformed into diaziridines in 2 or 3 steps by going through imines or oximes provided with leaving groups. The corresponding diaziridines are then oxidized into diazirines via an oxidant (Hill, J. R.; Robertson, A. A. B. J. Med. Chem. 2018, 61, 6945-6963).

Diazirines have been known since the 1980s as being very reactive carbene precursors. This property has enabled the increasing use of diazirines as photoaffinity labelling and photo-crosslinking reagents, the generated carbene rapidly forming a covalent bond with a nearby atom. These methods make it possible to identify active sites and study protein-protein interactions. Diazirines as photoactive probes have been widely used, for example for the design of organic electronic components (WO2016-049123), in cosmetic processes for the treatment of human body odors (WO2016-096897) and wrinkle reduction treatment (WO2010-076490), photocages for storing fragrances (WO2017-045891) or even as bioadhesives (WO2014-081391).

Diazirines labelled with nitrogen-15, denoted ¹⁵N₂-diazirines, can be used in hyperpolarization, a promising NMR technique in MRI imaging. [Theis, T.; Ortiz Jr., G. X.; Logan, A. W. J.; Clayton, K. E.; Feng, Y.; Huhn, W. P.; Blum, V.; Malcolmson, S. J.; Chekmenev, E. Y.; Wang, Q.; Warren, W. S. Science Advances, 2016, 2, e1501438]. Hyperpolarization of ¹⁵N₂-diazirines uses the SABRE-SHEATH (Signal Amplification by Reversible Exchange—Shield Enables Alignment Transfer to Heteronuclei) technique that is performed with an organometal catalyst based on iridium and parahydrogen (pH₂; available from molecular hydrogen H₂). This polarization is transferred to ¹⁵N₂-diazirines via the scalar couplings existing between the pH₂ and the nitrogen atoms of diazirine, following reversible ligand exchanges. This is then reflected by a spectacular increase in intensity of the NMR signal of nitrogen-15 (up to around 15,000 fold). In addition, this hyperpolarization can also be transferred to the proton, which makes it possible to consider its use in MRI, particularly in vivo [Shen, K.; Logan, A. W. J.; Colell, J. F. P.; Bae, J.; Ortiz Jr., G. X.; Theis, T.; Warren, W. S.; Malcolmson, S. J.; Wang, Q. Angew. Chem. Int. Ed. 2017, 56, 12112]. The introduction of the diazirine group onto molecules with a biological activity would thus allow use as a molecular marker for in-vivo MRI diagnosis (for diseases such as Alzheimer's disease and cancer), MRI being a very widespread imaging technique (approximately 820 devices in France), while avoiding positron emission tomography (PET) examinations, a very expensive technique using ionizing radiation and much less widespread (approximately 120 devices in France).

Diazirine synthesis is generally done in two or three steps, with low overall yields and has major drawbacks such as the use of hazardous reagents and restrictive reaction conditions such as, for example, the use of liquid ammonia (gaseous ammonia condensed at a temperature of −78° C.).

It appears that a single synthesis of ¹⁵N₂-diazirine has been done in three steps by using ¹⁵N-HOSA (hydroxylamine-O-sulfonic acid) and a carbonyl derivative, with only 18% overall yield. ¹⁵N-HOSA is itself prepared from ¹⁵N-hydroxylamine (¹⁵NH₂OH) and chlorosulfonic acid (CISO₃H) [Theis, T.; Ortiz Jr., G. X.; Logan, A. W. J.; Clayton, K. E.; Feng, Y.; Huhn, W. P.; Blum, V.; Malcolmson, S. J.; Chekmenev, E. Y.; Wang, Q.; Warren, W. S. Science Advances, 2016, 2, e1501438].

Surprisingly, the inventors have developed a one-pot diazirine synthesis from natural or unnatural amino acids, or imines previously formed from carbonyl compounds. Diazirines are obtained with good yields up to 99%. These methodologies can also be applied to the formation of diazirines enriched in nitrogen-15, or ¹⁵N₂-diazirines, also with high yields (ranging from 47% to 91%) by using an ammonia solution enriched in nitrogen-15 (¹⁵NH₃) formed beforehand.

Recently, Zakarian, A. et al. (J. Am. Chem. Soc. 2018, 140, 6027-6032) have developed a synthesis for pure liquid ¹⁵NH₃ from ammonium chloride, itself enriched in nitrogen-15 (¹⁵NH₄Cl). However, this method has the disadvantage of the ammonia changing to a gaseous state which necessitates recondensing it at a very low temperature (−78° C.).

Conversely, the inventors have also developed a synthesis for ¹⁵NH₃ in solution in an alcohol, typically methanol, from ¹⁵NH₄Cl, under very simple reaction conditions.

### SUMMARY OF THE INVENTION

The present invention relates to a one-pot synthesis method for a diazirine, wherein the nitrogen atoms each correspond, independently of one another, to the ¹⁴N isotope or to the ¹⁵N isotope, by reacting an amino acid or an imine with ammonia of formula ¹⁴NH₃ or ¹⁵NH₃ and an oxidant containing a hypervalent iodine atom.

A second subject of the invention concerns a synthesis method for ammonia enriched in nitrogen-15 of the formula ¹⁵NH₃ comprising the following steps:
(a′) reaction of ammonium chloride enriched with nitrogen-15, of formula ¹⁵NH₄Cl, with an alcoholate of formula R^{x}O-L in corresponding anhydrous alcohol of formula R^{x}OH,
(b′) optionally, elimination of the salt of formula L-Cl formed, advantageously by centrifugation.

The present invention also relates to diazirines derived from amino acids obtainable or directly obtained by the method of the invention.

The invention also relates to diazirines of formula (I′) enriched in nitrogen-15 and their use in hyperpolarization.

The present invention also relates to the use of diazirines derived from amino acids obtainable or directly obtained by the method of the invention and diazirines of formula (I′) in photoaffinity labelling.

### DETAILED DESCRIPTION OF THE INVENTION

The present invention concerns firstly a one-pot synthesis method for diazirine, that is or is not enriched in nitrogen-15, from an amino acid or an imine, corresponding to the reaction of the amino acid or imine with ammonia, that may or may not be enriched in nitrogen-15, and an oxidant containing a hypervalent iodine atom.

Within the meaning of the present invention, “one-pot synthesis” means that the synthesis of the diazirines of the invention from amino acids or imines is done without isolation of reaction intermediates, such as, for example, the corresponding diaziridines. The synthesis of the diazirines of the invention is advantageously conducted in a single reaction solvent.

Within the meaning of the present invention, the term “diazirine” designates any heterocycle with three members having a nitrogen-nitrogen double bond and a sp³ carbon, which may or may not be enriched in nitrogen-15.

In the present invention, the term “enriched in nitrogen-15” means that the compound concerned has a proportion of ¹⁵N isotope clearly higher than the natural isotopic abundance (0.36%). In the present invention, the compounds enriched in nitrogen-15 typically have an incorporation rate comprised between 60.0 and 99.9%. This incorporation rate depends on the percentage of enrichment in nitrogen-15 of the reagents that are nitrogen-15 sources. Within the meaning of the present invention, these are mainly commercial amino acids enriched in nitrogen-15 and commercial ammonium chloride enriched in nitrogen-15. These reagents are typically enriched in nitrogen-15 to 99%. The terms “enriched in nitrogen-15”, “having a nitrogen-15 atom” “for which one nitrogen atom is a ¹⁵N atom” have the same meaning. Likewise, “nitrogen-15” and “¹⁵N” are equivalent. If it is not specified that the compound is enriched in nitrogen-15, then it only contains nitrogen-14.

In the case where the diazirine obtained is enriched in nitrogen-15, this means that at least one of the two nitrogens making up the 3-member heterocycle, preferably both of them, is a ¹⁵N isotope. Other nitrogens that may be present in the diazirine outside of the three-member heterocycle can also be nitrogen-15.

Within the meaning of the present invention, “amino acid” refers to any natural or unnatural amino acid, which may or may not be enriched in nitrogen-15, regardless of their stereochemistry. The term “amino acid” designates any chemical compound that has both at least one carboxylic acid and at least one amine function. The side chain of the amino acid can be protected or unprotected. At least one carboxylic acid function and at least one amine function within the amino acid are unprotected. In the context of the present invention, it is advantageously α-amino acid, i.e., an amino acid wherein the carboxylic acid function and the amine function are carried by the same carbon atom. Natural amino acids include the following compounds: L-alanine, L-asparagine, L-aspartic acid, L-glycine, L-glutamine, L-leucine, L-isoleucine, L-phenylalanine, L-tryptophan, L-valine, L-histidine, L-tyrosine, L-glutamic acid, L-arginine, L-lysine, L-serine, L-threonine, L-proline, L-methionine, L-cysteine. In a particular embodiment, the amino acid is enriched in nitrogen-15. When the amino acid is enriched in nitrogen-15, at least the nitrogen of the amine function linked to the carbon bearing the carboxylic function is the ¹⁵N isotope. Other nitrogens eventually present in the side chain of the amino acid can also be the ¹⁵N isotope.

“Hypervalent iodine” means an iodine atom, within a molecule, having more than eight electrons in its valence shell and therefore not conforming with the octet rule. The iodine is thus found either in a (+III) oxidation state and is then a λ³-iodane, or in a (+V) oxidation state and is then a λ⁵-iodane. In the present invention, the oxidant used advantageously comprises a λ³-iodane iodine atom. Said oxidant can be prepared and isolated upstream of the method or prepared in situ during the method of the invention from an iodine derivative in the presence of an oxidant or from an iodoso derivative. Preferably, the oxidant containing a hypervalent iodine atom used during the method of the invention is obtained upstream of the method.

Within the meaning of the present invention, “oxidant with hypervalent iodine” refers to a chemical compound with oxidizing power and containing a hypervalent iodine atom. Typically, the oxidant with hypervalent iodine has the role of oxidizing the reaction intermediates, such as diaziridines, to lead to the corresponding diazirines. It can also act as a catalyst of the intermediate reactions that occur during the diazirine synthesis of the invention from amino acids or imines. In one embodiment, the oxidant containing a hypervalent iodine atom can be chosen from λ³-iodane compounds. For example, the hypervalent iodine oxidant can be chosen among the following compounds:

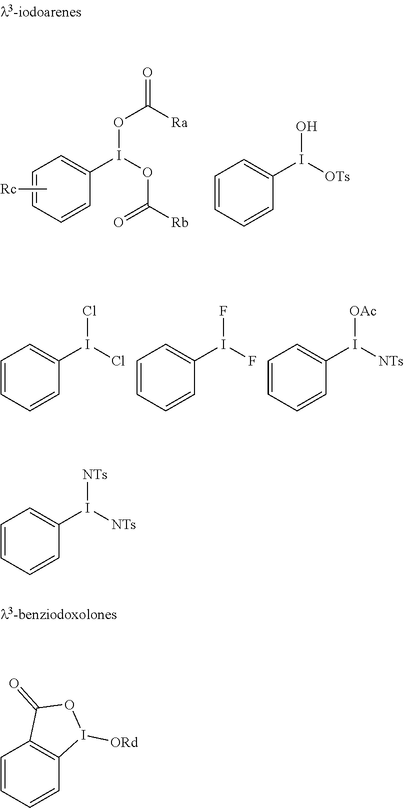

where Ra and Rb each represent, independently of one another, a C₁-C₆ alkyl, especially a methyl or a tert-butyl, or a C₁-C₆ haloalkyl, especially a trifluoromethyl,

Rc represents one or more substituents chosen from C₁-C₆ alkyl, especially a methyl group or an ethyl group, and halogen, especially Cl, Br or I, and

Rd represents a hydrogen, a C₁-C₆ alkyl, such as methyl, or an acetate group.

Preferably, the hypervalent iodine oxidant can be chosen among the following compounds:

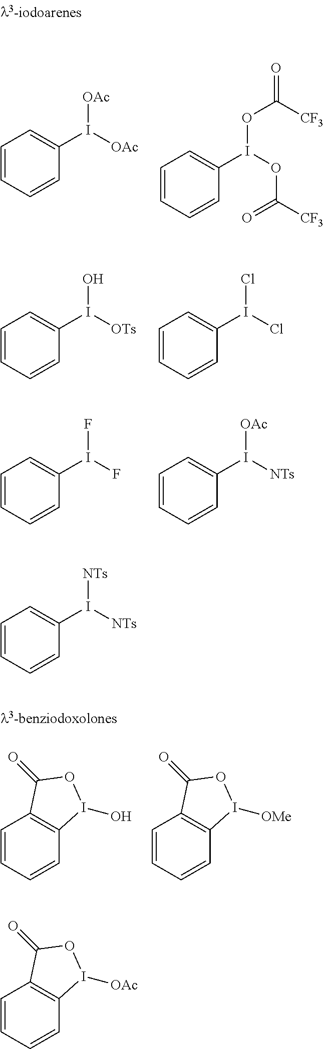

Still more preferably, the oxidant used is PIDA (phenyliodonium diacetate; PhI(OAc)₂] of the following formula:

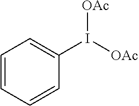 [SMILES: CC(=O)OI(OC(C)=O)c1ccccc1]

The ammonia source can be pure gaseous or liquid ammonia, ammonia in solution in a solvent such as methanol or water, or an ammonium of the general formula NH₄⁺X⁻, where X⁻ advantageously represents a counterion chosen from the group consisting in:

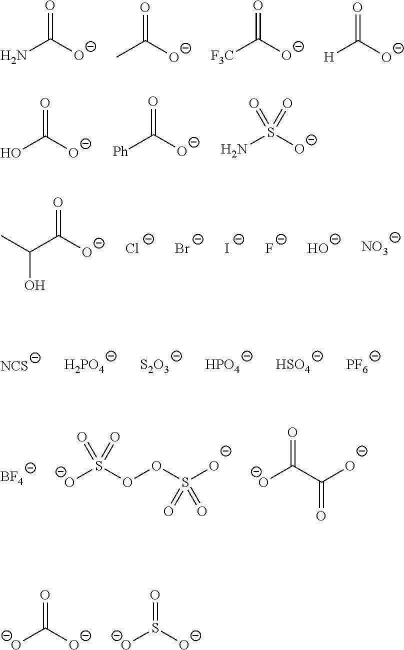

Preferably, the ammonia source is ammonia in solution in a solvent, preferably ammonia in solution in methanol.

The ammonia used may or may not be enriched in nitrogen-15. According to a preferred embodiment, the ammonia is enriched in nitrogen-15.

In another particular embodiment, the diazirine obtained by means of the method of the invention does not contain nitrogen-15. According to another embodiment, the diazirine obtained by means of the method of the invention is enriched in nitrogen-15 and contains one or two nitrogen-15 atom(s).

Advantageously, the present invention concerns a one-pot synthesis method for diazirine, which is or is not enriched in nitrogen-15, from an α-amino acid or an imine, in particular an α-amino acid, comprising the reaction of the α-amino acid or imine with ammonia, which is or is not enriched in nitrogen-15, and an oxidant containing λ³-iodane-type hypervalent iodine atom.

According to a preferred embodiment, the method of the invention makes it possible to synthesize a diazirine of formula (I) below:

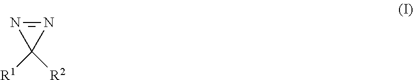

or a physiologically acceptable salt or solvate thereof, a stereoisomer or a mixture of stereoisomers in any proportions, wherein the nitrogen atoms each, independently of one another, correspond to the ¹⁴N isotope or the ¹⁵N isotope, and wherein R¹ represents H, V, W or V—W where:
V represents an aliphatic chain where up to 8, preferably 4, methylene units of the aliphatic chain are optionally replaced by O, C(O), S, S(O), S(O)₂, NR′ or SiR′R″; V is optionally substituted by 1 to 12 groups selected from OH, NR′R″, halogen, CN, oxo, (═NR′) and aryl,
W represents a cycloalkyl, an aryl, a heterocycle or a heteroaryl; W is optionally substituted by 1 to 4 groups selected from halogen, CN, NO₂, OH, NR′R″ and an aliphatic chain, where up to 4 methylene units of the aliphatic chain are optionally replaced by O, C(O), S, S(O), S(O)₂, NR′ or SiR′R″,
R′ and R″ each represent, independently of one another, H or an aliphatic chain, where up to 4 methylene units of the aliphatic chain are optionally replaced by O, C(O), S, S(O), S(O)₂, NR′ or SiR′R″, and
R² represents H or an aliphatic chain, where up to 4 methylene units of the aliphatic chain are optionally replaced by O, C(O), S, S(O), S(O)₂, NR′ or SiR′R″, said method comprising the following steps:
(a) Reaction of a compound of formula (II) below:

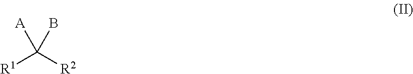

or a physiologically acceptable salt or solvate thereof, a tautomer, a stereoisomer or a mixture of stereoisomers in any proportions,

wherein A represents NH₂, the nitrogen atom being the ¹⁴N isotope or the ¹⁵N isotope, B represents COOH and R¹ and R² are as defined above,

or A-B forms the same imine group of formula ═NR³, and R³ represents V, W or V—W as defined above, with an oxidant comprising a hypervalent iodine atom of formula (III) below:

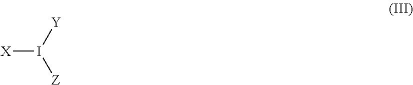

wherein X represents a (C₁-C₁₀)alkyl, an aryl or a heteroaryl; X being optionally substituted with 1 to 12 groups selected from OH, NR′R″, halogens, CN, oxo, (═NR′) and aryl,

Y and Z represent, independently of one another, OH, a halogen, NH₂, CN, a (C₁-C₁₀)alkyl group, a ((C₁-C₁₀)alkyl)-aryl group or a ((C₁-C₁₀)alkyl)-heteroaryl group, where up to 4 methylene units of said alkyl are optionally replaced by O, C(O), S(O)₂ or NH,

and in the presence of ¹⁴NH₃ or ¹⁵NH₃ ammonia, in a reaction solvent at a temperature advantageously comprised between 0° C. and ambient temperature, typically for a time period comprised between 1 h and 4 h.
(b) Advantageously, purification of the diazirine of formula (I) obtained in step (a), advantageously by chromatography or by distillation.

Physiologically acceptable salts of the compounds of the present invention comprise standard non-toxic salts of the compounds of the invention, such as those formed with organic or inorganic acids or organic or inorganic bases. Within the meaning of the present invention, salts formed from the compounds of the invention whose amine function is in an ammonium form and/or the acid function is in a carboxylate form can be particularly mentioned. Examples include counterions, chlorine, bromine, fluorine, nitrate or bicarbonate forming ammonium salts, and counterions derived from alkaline metals such as Na⁺, Li⁺ and K⁺ forming carboxylate salts.

These salts can be synthesized from compounds of the invention containing a basic or acid part and the corresponding acids or bases according to standard chemical methods. Acceptable solvates for the compounds according to the present invention include standard solvates such as those formed during the last step of the preparation method for the compounds according to the method with the reaction solvent(s). Solvates formed with water (commonly called hydrates) or with methanol or ethanol can be mentioned as examples.

Within the meaning of the present invention, “aliphatic chain” designates a linear or branched hydrocarbon chain, completely saturated or containing one or more unsaturations, but not aromatic. An aliphatic chain according to the present invention advantageously comprises from 1 to 18 carbon atoms, preferably from 1 to 12 carbon atoms, more preferably from 1 to 10 carbon atoms. According to the present invention, the term “aliphatic chain” includes substituted or unsubstituted, linear or branched, alkyl, alkenyl or alkynyl groups.

Within the meaning of the present invention, “alkyl” group designates a saturated, linear or branched hydrocarbon chain. Examples include methyl, ethyl, propyl, isopropyl, butyl, isobutyl, sec-butyl, tert-butyl, pentyl or hexyl groups.

Within the meaning of the present invention “alkenyl group” designates a linear or branched hydrocarbon chain bearing one or more double bonds.

Within the meaning of the present invention “alkynyl group” designates a linear or branched hydrocarbon chain bearing at least one triple bond. Ethynyl or propynyl groups can be named as an example.

Within the meaning of the present invention, “aryl” designates an aromatic hydrocarbon group, preferably containing 6 to 10 carbon atoms and comprising one or more fused rings. It will advantageously be a phenyl or naphthyl group.

Within the meaning of the present invention “cycloalkyl” designates a nonaromatic hydrocarbon ring, completely saturated or containing one or more unsaturations, advantageously comprising 3 to 10 members. The term includes fused, spiro, or bridged polycyclic compounds. Examples of cycloalkyls are cyclopropenyl, cyclohexyl or cyclobutyl.

Within the meaning of the present invention, “heterocycle” designates a nonaromatic ring, completely saturated or containing one or more unsaturations, advantageously comprising 3 to 10 members, wherein one or more carbon atoms, advantageously 1 to 4 and still more advantageously 1 or 2, are each replaced by a heteroatom chosen from among sulfur, nitrogen and oxygen atoms. The term includes fused, spiro, or bridged polycyclic compounds. Advantageously, it will be a monocyclic or a bicyclic compound wherein 1 or 2 carbon atoms are each replaced by a heteroatom chosen from sulfur, nitrogen and oxygen atoms, preferably chosen from nitrogen and oxygen atoms, such as nitrogen. Examples of heterocycles are piperidinyl, piperizinyl, pyrrolidinyl, pyrazolidinyl, imidazolidinyl, azepanyl, thiazolidinyl, isothiazolidinyl, oxazocanyl, thiazepanyl and benzimidazolonyl.

Within the meaning of the present invention, “heteroaryl” designates an aromatic group comprising one or more, especially 1 or 2, fused hydrocarbon rings, wherein one or more carbon atoms, advantageously 1 to 4 and still more advantageously 1 or 2, are each replaced by a heteroatom chosen from among sulfur, nitrogen and oxygen atoms and wherein each ring advantageously comprises 5 to 7 members, preferably 5 or 6 members. Advantageously, it will be an aromatic group comprising 1 or 2 fused hydrocarbon rings, each ring having 5 or 6 members, wherein 1 or 2 carbon atoms are each replaced by a heteroatom chosen from sulfur, nitrogen and oxygen atoms, preferably chosen from nitrogen and oxygen atoms, such as nitrogen. Examples of heteroaryl groups are furyl, thienyl, pyrrolyl, oxazolyl, isoxazolyl, thiazolyl, isothiazolyl, imidazolyl, pyrazolyl, oxadiazolyl, thiadiazolyl, triazolyl, tetrazolyl, pyridyl, pyridazinyl, pyrimidinyl, pyrazinyl, triazinyl, quinolyl, quinoxalyl or indolyl. It will especially be a pyridyl, indolyl or imidazolyl group.

Within the meaning of the present invention “halogen atom” designates fluorine, chlorine, bromine and iodine atoms.

Within the meaning of the present invention, “unsaturated” means that the hydrocarbon chain can have one or more unsaturations, advantageously one.

Within the meaning of the present invention, “unsaturation” designates a carbon-carbon double or triple bond (C═C or C≡C).

Within the meaning of the present invention, “stereoisomer” means a geometric isomer or an optical isomer.

Geometric isomers result from the different position of substituents on a double bond that can have a Z or E configuration.

Optical isomers result from the different position in space of substituents on a carbon atom with 4 different substituents. This carbon is therefore a chiral or asymmetric center. Optical isomers include diastereoisomers and enantiomers. Optical isomers that are mirror images of one another but cannot be superimposed are called “enantiomers”. Optical isomers that are not superimposable or mirror images are called “diastereomers”.

A mixture containing equal quantities of two individual enantiomer forms of opposite chirality is called a “racemic mixture”.

Within the meaning of the present invention, “chiral group” designates a group that is not superimposable on its mirror image. Such a chiral group will particularly be able to comprise an asymmetric carbon atom, i.e., a carbon atom substituted by four different substituents (including hydrogen).

Within the meaning of the present invention, the stereoisomerism of the compounds can be induced by side chains R¹ and/or R².

When an aryl is said to be substituted by an aliphatic chain, within the meaning of the present invention, it means that the aliphatic chain can be located between said aryl and the rest of the molecule. For example, when R³ represents an aryl substituted by an aliphatic chain, this can signify that the aliphatic chain is directly bound to the nitrogen atom bearing the R³ radical and said aliphatic chain bears an aryl. Examples of the groups are benzyl and tosyl, where the methylene group of the aliphatic chain is replaced by a sulfone group. An aryl substituted by an aliphatic chain also designates the case where the aryl is directly bound to the rest of the molecule and bears an aliphatic chain. Within the meaning of the present invention, the term “ambient temperature” used in the following designates a temperature ranging from 18° C. to 30° C., preferably ranging from 20° C. to 25° C., more preferably 22° C.

Preferably, R¹ represents H, V, W or V—W where:
V represents an aliphatic chain where up to 4 methylene units of the aliphatic chain are optionally replaced by O, C(O), S, S(O), S(O)₂, NR′ or SiR′R″; V is optionally substituted by 1 to 12 groups selected from OH, NR′R″, halogen, CN, oxo, (═NR′) and aryl,
W represents a cycloalkyl, an aryl, a heterocycle or a heteroaryl; W is optionally substituted by 1 to 4 groups selected from halogen, CN, NO₂, OH, NR′R″ and an aliphatic chain, where up to 4 methylene units of the aliphatic chain are optionally replaced by O, C(O), S, S(O), S(O)₂, NR′ or SiR′R″, R′ and R″ being such as defined above.

In a preferred embodiment, R² represents H or an alkyl group, preferably a methyl. Advantageously R² is a hydrogen atom.

In a preferred embodiment, R³ represents an aliphatic chain, a cycloalkyl or an aryl; wherein up to 4 methylene units of the aliphatic chain are optionally replaced by O, C(O), S, S(O), S(O)₂, NR′ or SiR′R″, and wherein said aryl is optionally substituted by 1 to 4 groups selected from among halogens, CN, NO₂, OH, NR′R″ and an aliphatic chain such as defined above.

Preferably, R³ represents a benzyl, tosyl, cyclohexyl, hydroxyl or alkyl group such as a tert-butyl group.

Preferably, R³ is a tert-butyl or tosyl group.

In a preferred embodiment, X is an aryl, for example a phenyl, and/or Y and Z are preferably identical, and more preferably, Y and Z represent a halogen, for example a chlorine or a fluorine, an OH, NH₂, CN, O—(C₁-C₆)alkyl, O—(C₁-C₆)-perfluoroalkyl, O-aryl, O—S(O)₂—(C₁-C₆)alkyl, O—S(O)₂-heteroaryl, NH—SO₂—(C₁-C₆)alkyl, NH—S(O)₂-heteroaryl, N(SO₂—(C₁-C₆)alkyl)₂, N(S(O)₂-heteroaryl)₂ group or an ester O(CO)(C₁-C₆)alkyl, O(CO) (C₁-C₆)perfluoroalkyl, O(CO)aryl group, preferably an ester O(CO)(C₁-C₆)alkyl group.

Preferably, the oxidant is phenyliodonium diacetate, designated PIDA, for which X is a phenyl and Y and Z are both O—C(O)—CH₃.

In a preferred embodiment, the solvent used is a solvent advantageously chosen from alcohols, acetonitrile, THF, toluene, dichloromethane, ethyl acetate, DMSO, acetone, pyridine and DMF. The reaction advantageously takes place in an alcohol such as methanol, ethanol, propan-1-ol, n-butanol, tert-butanol, pentan-1-ol, hexan-1-ol, preferably methanol.

Advantageously, the reaction solvent is the ammonia source.

Step (a)

Advantageously the ammonia is used in excess relative to the compound of formula (II), preferably in an amount of 15 to 20 equivalents, preferably 17 to 18 equivalents. In particular, the ammonia is used in an amount of 17.5 equivalents relative to the compound of formula (II).

According to one embodiment, the compound of formula (II) is added to an ammonia solution in a solvent, preferably an ammonia solvent in methanol.

The oxidant comprising a hypervalent iodine atom is advantageously used in excess relative to the compound of formula (II) in an amount of 2 to 5 equivalents, preferably 3 equivalents.

In one embodiment, the oxidant comprising the hypervalent iodine atom is added to the reaction medium made up of ammonia, the compound of formula (II) and the solvent, preferably when the reaction medium is at a temperature of 0° C.

A single oxidant addition is advantageously performed during the method for obtaining diazirines from compounds of formula (II).

Typically, during step (a), the temperature of the reaction medium is, firstly, maintained at 0° C. for a period comprised between 15 minutes and 45 minutes, preferably 30 minutes, after the addition of the oxidant comprising a hypervalent iodine atom, then it is raised to ambient temperature, and typically maintained at this temperature, typically for a time comprised between 1 hour and 3 hours, preferably for 1 hour and 30 minutes.

Step (b)

The diazirine obtained in step (a) is advantageously purified, preferably by chromatography or distillation.

Within the meaning of the present invention, “chromatography” designates a purification technique for compounds based on the difference in affinity of the compounds with the mobile phase and the stationary phase. There are different types of chromatography: adsorption, exclusion, ion exchange or partition chromatography. Advantageously, in the present invention, the compounds will be separated by adsorption chromatography. The mobile phase can be a liquid, a gas or a supercritical fluid. Preferably, the mobile phase will be a liquid, preferably a mixture of solvents of different polarities, for example two solvents, chosen from among pentane, ethyl acetate, methanol, dichloromethane, ethanol or diethyl ether. The stationary phase can be paper, modified or unmodified silica, polymers, alumina, etc. Advantageously, in the context of the present invention, the stationary phase will be silica.

Preferably, the chromatography will be silica gel chromatography.

In one embodiment, the compound of formula (II) is an amino acid of formula (IIa) below:

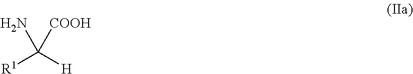

or a physiologically acceptable salt or solvate thereof, a stereoisomer or a mixture of stereoisomers in any proportions

wherein the nitrogen atom of the amine function bound to the carbon also bearing the carboxylic acid function COOH, is either the ¹⁴N isotope or the ¹⁵N isotope, and

R¹ represents H, V, W or V—W where:
V represents an aliphatic chain where up to 8, preferably 4, methylene units of the aliphatic chain are optionally replaced by O, C(O), S, S(O), S(O)₂, NR′ or SiR′R″; V is optionally substituted by 1 to 12 groups selected from OH, NR′R″, halogen, CN, oxo, (═NR′) and aryl,
W represents a cycloalkyl, an aryl, a heterocycle or a heteroaryl; W is optionally substituted by 1 to 4 groups selected from halogen, CN, NO₂, OH, NR′R″ and a (C₁-C₁₀) aliphatic chain, where up to 4 methylene units of the (C₁-C₁₀) aliphatic chain are optionally replaced by O, C(O), S, S(O), S(O)₂, NR′ or SiR′R″,
R′ and R″ each represent, independently of one another, H or an aliphatic chain, where up to 4 methylene units of the aliphatic chain are optionally replaced by O, C(O), S, S(O), S(O)₂, NR′ or SiR′R″.

In a preferred embodiment, compound (IIa) is one of the following amino acids: L-aspartic acid, L-asparagine, L-glutamine, L-glycine, L-alanine, L-valine, L-isoleucine, L-leucine, L-phenylalanine, L-tryptophan, 4-methyl-L-phenylalanine, 4-iodo-L-phenylalanine L-histidine, L-tyrosine, L-glutamic acid.

In a particular embodiment, compound (IIa) corresponds to natural amino acids. Preferably, compound (IIa) corresponds to amino acids that make it possible to obtain non-volatile diazirines, such as the following natural amino acids: L-histidine, L-tyrosine or L-glutamic acid.

In a particular embodiment of the invention, the method comprises a preliminary protection step for compound (IIa).

“Protection step” means the placement of a protective group on a chemical function present in the R¹ group of compound (IIa). The term “protection step” can also designate a reversible chemical reaction to inhibit the reactivity of a chemical function under the reaction conditions of the method of the invention, such as, for example, a step of oxidation, reduction or amidation.

Within the meaning of the present invention, “protective group” means a group that protects a chemically reactive function against undesirable reactions, such as the groups described by T.W. Greene, “Protective Groups in Organic Synthesis”, (John Wiley & Sons, New York (1981)) and Harrison et al. “Compendium of Synthetic Organic Methods”, Vols. 1 to 8 (J. Wiley & sons, 1971 to 1996).

Within the meaning of the present invention, the term “chemical function” designates any chemically reactive entity that can react during the method of the invention. It can be an atom, a collection of atoms, for example a saturated, unsaturated or aromatic ring or heterocycle.

Preferably, the preliminary protection step is done on compound (IIa) wherein R¹ comprises a function chemically incompatible with the reagents used in the method of the invention, such as OH, NH₂, C(O)NH₂, C(O)OH, SH, a guanidine function, an indole ring or a thioether function.

More particularly, the preliminary protection step is performed on compound (IIa) wherein R¹ is the side chain of the following amino acids: lysine, arginine, serine, threonine, methionine, tryptophan and cysteine.

The protective groups according to the present invention include, in particular, a tert-butoxycarbonyl group, commonly abbreviated Boc, a benzyl group (Bn), a trityl group (Tr), a carboxybenzyl group (CBz), acyl groups such as the acetate group (Ac) or silylated protective groups, such as tert-butyldiphenylsilyl (TBDPS).

Advantageously, the protection step of a compound (IIa) whose R¹ group has a thioether function is oxidation of this atom into sulfoxide. Advantageously, a thiol function is protected in the form of thioethers, for example with a trityl.

Advantageously, an NH₂ function present in the R₁ group of compound (IIa) is protected in the amide or carbamate form, for example using an acetate group, a Boc group or a CBz group.

Advantageously, an OH function present in the R¹ group of compound (IIa) is protected in the form of an ether, especially using a benzyl, and more particularly silylated ethers, for example using TBDPS.

Advantageously, an indole function in the R¹ group of compound (IIa) is protected by introduction of a Boc group.

According to a preferred embodiment, compound (IIa) corresponds to the following protected amino acids: N_{ε}-acetyl-L-lysine, N_{ε}-benzyloxycarbamate-L-lysine, L-methionine sulfoxide, S-trityl-L-cysteine, S-benzyl-L-cysteine, O-benzyl-DL-serine, O-tert-butyldiphenylsilyl-DL-serine, O-tert-butyldiphenylsilyl-DL-threonine, 1-(tert-butoxycarbonyl)-L-tryptophan, L-theanine, γ-benzyl-L-glutamic acid.

In a particular embodiment, compound (IIa) undergoes functional modification to reduce the volatility of the corresponding diazirines. Said functional modification typically corresponds to the introduction of groups that will increase the molecular mass and/or the polarity of the compound to make it less volatile. Groups that make it possible to reduce the volatility of compounds include, for example, halogens, in particular iodine, or isopropyl or tert-butyl groups. The introduction of an iodine atom is particularly interesting because it makes it possible efficiently to carry out subsequent functionalizations by organometal coupling.

Advantageously, compound (IIa) is la 4-iodophenyl-alanine.

In a particular embodiment of the invention, the diazirine according to the invention, advantageously of formula (I), enriched in nitrogen-15, is obtained by means of the method according to the invention from amino acid (IIa) enriched in nitrogen-15 and ammonia enriched in nitrogen-15.

The diazirine according to the invention, advantageously of formula (I), enriched in nitrogen-15 obtained from amino acid (IIa) enriched in nitrogen-15 and ammonia enriched in nitrogen-15 has a nitrogen-15 incorporation rate greater than 99%.

According to another embodiment, the diazirine according to the invention, advantageously of formula (I), enriched in nitrogen-15, is obtained by means of the method according to the invention from amino acid (IIa) not enriched in nitrogen-15 and ammonia enriched in nitrogen-15.

The diazirine according to the invention, advantageously of formula (I), enriched in nitrogen-15 obtained from amino acid (IIa) not enriched in nitrogen 15 and ammonia enriched in nitrogen-15 has a nitrogen-15 incorporation rate comprised between 60% and 90%.

According to another embodiment, the compound of formula (II) is an imine of formula (IIb) below:

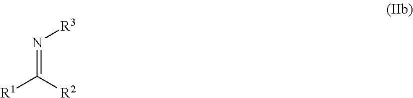

or a physiologically acceptable salt or solvate thereof, a tautomer, a stereoisomer or a mixture of stereoisomers in any proportions, wherein:

R¹ represents H, V, W or V—W where:
V represents an aliphatic chain where up to 8, preferably 4, methylene units of the aliphatic chain are optionally replaced by O, C(O), S, S(O), S(O)₂, NR′ or SiR′R″; V is optionally substituted by 1 to 12 groups selected from OH, NR′R″, halogen, CN, oxo, (═NR′) or aryl,
W represents a cycloalkyl, an aryl, a heterocycle or a heteroaryl; W is optionally substituted by 1 to 4 groups selected from halogen, CN, NO₂, OH, NR′R″ and an aliphatic chain, where up to 4 methylene units of the aliphatic chain are optionally replaced by O, C(O), S, S(O), S(O)₂, NR′ or SiR′R″,
R′ and R″ each represent, independently of one another, H or an aliphatic chain, where up to 4 methylene units of the aliphatic chain are optionally replaced by O, C(O), S, S(O), S(O)₂, NR′ or SiR′R″,
R² represents H or an aliphatic chain, where up to 4 methylene units of the aliphatic chain are optionally replaced by O, C(O), S, S(O), S(O)₂, NR′ or SiR′R″, and

R³ represents V, W or V—W such as defined above.

In a preferred embodiment, in compound (IIb), R¹ represents a (C₁-C₄) aliphatic chain as defined above. Preferably, R¹ comprises a carboxylic acid function COOH. In particular, R¹ represents an alkyl-COOH group, for example CH₂CH₂COOH.

In another preferred embodiment, in compound (IIb), when R¹ is an aryl, it is advantageously not substituted or substituted by 1 to 4 groups selected from CN, NO₂, OH, NR′R″ and an aliphatic chain, where up to 4 methylene units of the aliphatic chain are optionally replaced by C(O)O, S, S(O), S(O)₂, NR′ or SiR′R″, Advantageously, R′ is an aryl, preferably a phenyl substituted by a CN, NO₂, C(O)—(C₁-C₄ alkyl) or C(O)O—(C₁-C₄alkyl) group. More preferably, R′ is a phenyl substituted by a nitrile group (CN) or an NO₂ group.

According to one embodiment, R² represents H or an alkyl group, preferably a methyl.

In a preferred embodiment, R³ represents an aliphatic chain, a cycloalkyl or an aryl; wherein up to 4 methylene units of the aliphatic chain are optionally replaced by O, C(O), S, S(O), S(O)₂, NR′ or SiR′R″, and wherein said aryl is optionally substituted by 1 to 4 groups selected from halogen, CN, NO₂, OH, NR′R″ and an aliphatic chain such as defined above.

Advantageously, R³ represents a benzyl, tosyl, cyclohexyl, hydroxyl or alkyl group such as a tert-butyl group. Preferably, R³ is a tert-butyl or tosyl group.

According to one embodiment of the invention, the imine of formula (IIb) is obtained beforehand from a carbonyl compound of formula (IV) below:

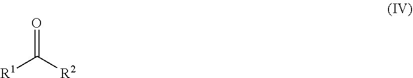

or a physiologically acceptable salt thereof, a tautomer, a stereoisomer or a mixture of stereoisomers in any proportions

wherein R¹ and R² are as defined above.

Preferred embodiments concerning R¹ and R² in compound (IV) are identical to those relating to compound (IIb).

The reaction to obtain the imine of formula (IIb) advantageously comprises the reaction of the carbonyl compound of formula (IV) with an amine of formula (V) below:

R³—NH₂ (V)

wherein R³ is as defined above. Preferably, R³ represents a benzyl, tosyl, cyclohexyl, hydroxyl or alkyl group such as a tert-butyl group. Preferably, R³ is a tert-butyl or tosyl group.

According one embodiment, the diazirine according to the invention, advantageously of formula (I), enriched in nitrogen-15, is obtained by means of the method according to the invention from imine (IIb) not enriched in nitrogen-15 and ammonia enriched in nitrogen-15.

The present invention also concerns a synthesis method for ammonia enriched in nitrogen-15 of the formula ¹⁵NH₃ comprising the following steps:
(a′) reaction of ammonium chloride enriched with nitrogen-15, of formula ¹⁵NH₄Cl with an alcoholate of formula R^{x}O-L in corresponding anhydrous alcohol of formula R^{x}OH,
where R^{x} is an alkyl group, preferably a C₁-C₆alkyl group, more preferably R^{x} is a methyl, and L is a counterion chosen from Na⁺, K⁺ and Li⁺, preferably Na⁺,
(b′) optionally, elimination of the L-Cl salt formed, advantageously by centrifugation.

The ammonium chloride used is typically enriched in nitrogen-15 to 99%.

Advantageously, the ammonium chloride enriched in nitrogen-15 and the alcoholate of formula R^{x}O-L are used in equivalent amounts. The temperature of the method is preferably comprised between 0° C. and ambient temperature and the reaction time is comprised between 2 hours 30 minutes and 3 hours.

Preferably, the alcoholate of formula R^{x}O-L is added to a solution of ammonium chloride enriched in nitrogen-15 in the corresponding anhydrous alcohol R^{x}OH at a temperature of 0° C. In particular, the alcoholate of formula R^{x}O-L is added to a solution of ammonium chloride enriched in nitrogen-15 in the corresponding anhydrous alcohol R^{x}OH in several portions, for example five portions, over a certain period of time, typically comprised between 15 minutes and 1 hour, preferably over a period of 30 minutes. In a particular embodiment, the reaction medium composed of the alcoholate of formula R^{x}O-L and a solution of ammonium chloride enriched in nitrogen-15 in the corresponding anhydrous alcohol R^{x}OH is typically stirred at ambient temperature, advantageously for a period of time comprised between 1 hour and 30 minutes and 3 hours, preferably for 2 hours.

In a preferred embodiment, the anhydrous alcohol used is chosen from methanol, ethanol, trifluoroethanol, propan-1-ol, propan-2-ol, hexafluoropropan-2-ol, n-butanol, tert-butanol, pentan-1-ol, pentan-2-ol, pentan-3-ol, hexan-1-ol, hexan-2-ol and hexan-3-ol, preferably, the anhydrous alcohol according to the invention is methanol.

Consequently, the alcoholate of formula R^{x}O-L used is advantageously chosen from among lithium, sodium or potassium alcoholates of the anhydrous alcoholates mentioned above, preferably the alcoholate according to the invention is sodium methanolate.

When the method for preparing diazirine according to the invention uses ammonia enriched in nitrogen-15, it is preferably prepared via the method previously described in the present invention.

The present invention also concerns diazirines according to the invention, advantageously of formula (I), not enriched in nitrogen-15, derived from amino acids, obtainable or directly obtained via the method of the invention for diazirines according to the invention.

Diazirines of formula (I) not enriched in nitrogen-15 according to the invention result from natural or unnatural amino acids, protected or not.

Advantageously, the diazirines according to the invention obtainable or directly obtained via the method for obtaining diazirines according to the invention are of formula (I) below:

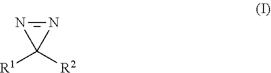

or a physiologically acceptable salt or solvate thereof, a stereoisomer or a mixture of stereoisomers in any proportions, wherein the nitrogen atoms each correspond, independently of one another, to the ¹⁴N isotope or the ¹⁵N isotope,

and wherein R¹ represents H, V, W or V—W where:
V represents an aliphatic chain where up to 8, preferably 4, methylene units of the aliphatic chain are optionally replaced by O, C(O), S, S(O), S(O)₂, NR′ or SiR′R″; V is optionally substituted by 1 to 12 groups selected from OH, NR′R″, halogen, CN, oxo, (═NR′) and aryl,
W represents a cycloalkyl, an aryl, a heterocycle or a heteroaryl; W is optionally substituted by 1 to 4 groups selected from halogen, CN, NO₂, OH, NR′R″ and an aliphatic chain, where up to 4 methylene units of the aliphatic chain are optionally replaced by O, C(O), S, S(O), S(O)₂, NR′ or SiR′R″,
R′ and R″ each represent, independently of one another, H or an aliphatic chain, where up to 4 methylene units of the aliphatic chain are optionally replaced by O, C(O), S, S(O), S(O)₂, NR′ or SiR′R″, and

R² represents H or an aliphatic chain, where up to 4 methylene units of the aliphatic chain are optionally replaced by O, C(O), S, S(O), S(O)₂, NR′ or SiR′R″. Advantageously, R² represents a hydrogen or a (C₁-C₆) alkyl group, such as methyl, preferably a hydrogen.

In diazirines of formula (I), when R² is a methyl group, R¹ is preferably a phenyl substituted by a CN, NO₂, C(O)—(C₁-C₄ alkyl) or C(O)O—(C₁-C₄ alkyl) group. More preferably, R¹ is a phenyl substituted by a nitrile (CN) or NO₂ group.

Diazirines of formula (I) not enriched in nitrogen-15 obtainable or directly obtained by the method of the invention advantageously correspond to the following compounds:

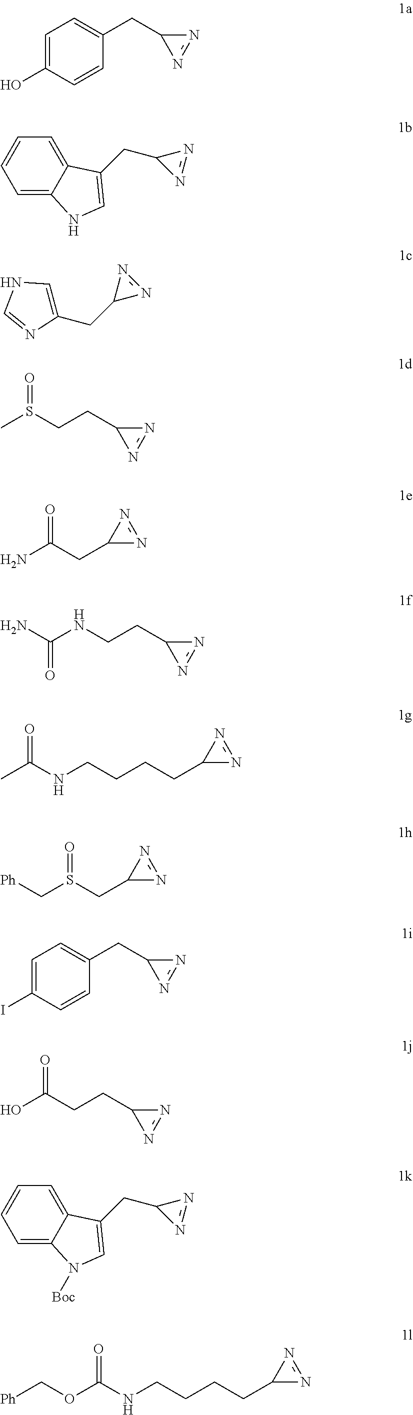

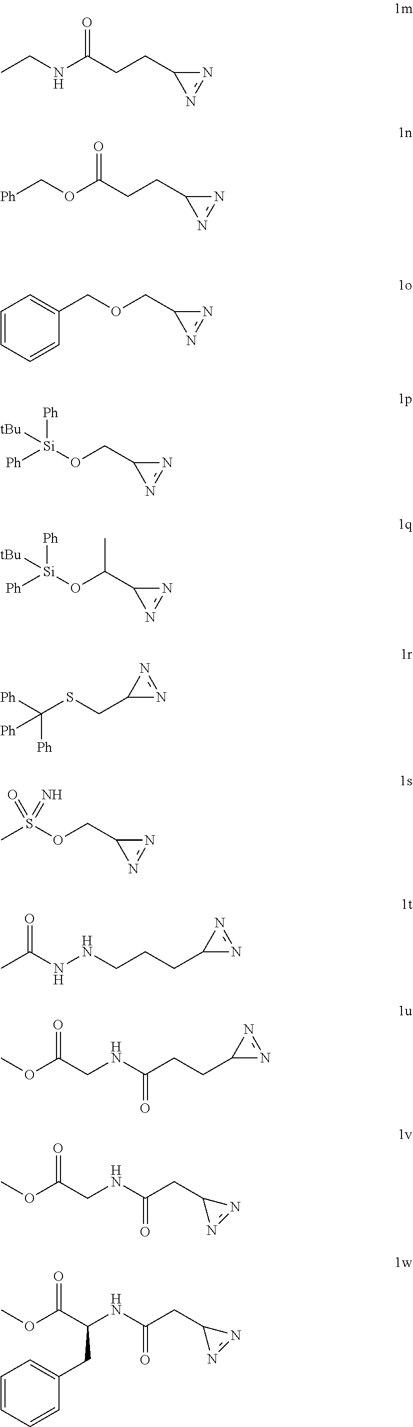

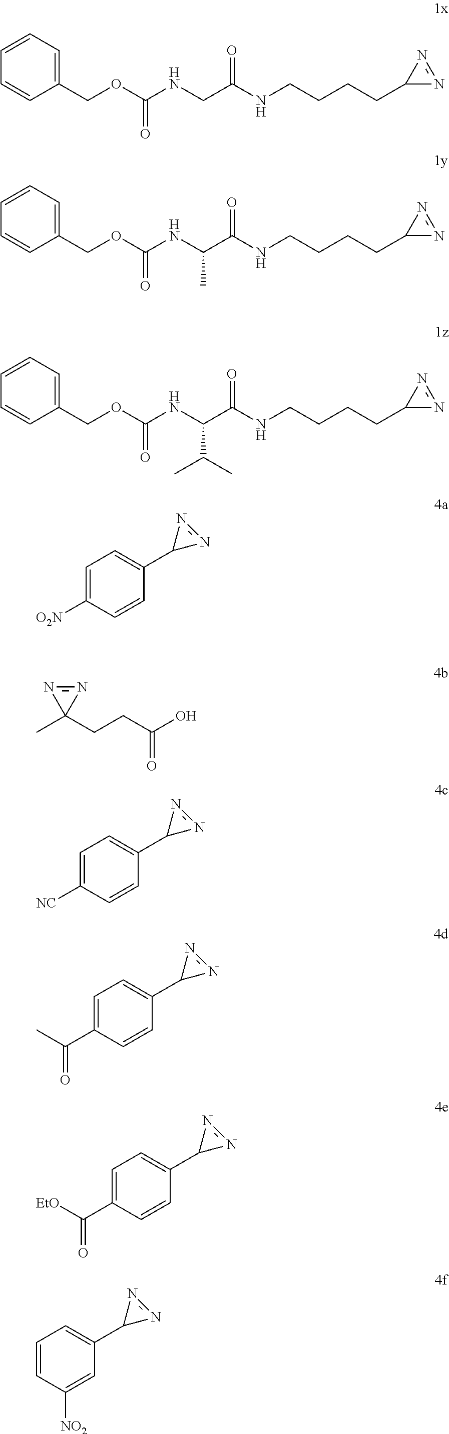

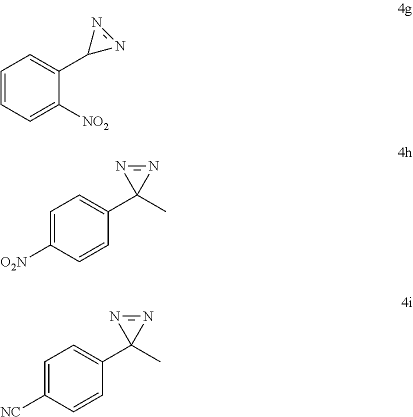

In a particular embodiment, the diazirines of formula (I) not enriched in nitrogen-15 according to the invention are obtainable or directly obtained by the method of the invention wherein compound (II) is of formula (IIb). According to this embodiment, the diazirines of formula (I) according to the invention advantageously correspond to the following compounds:

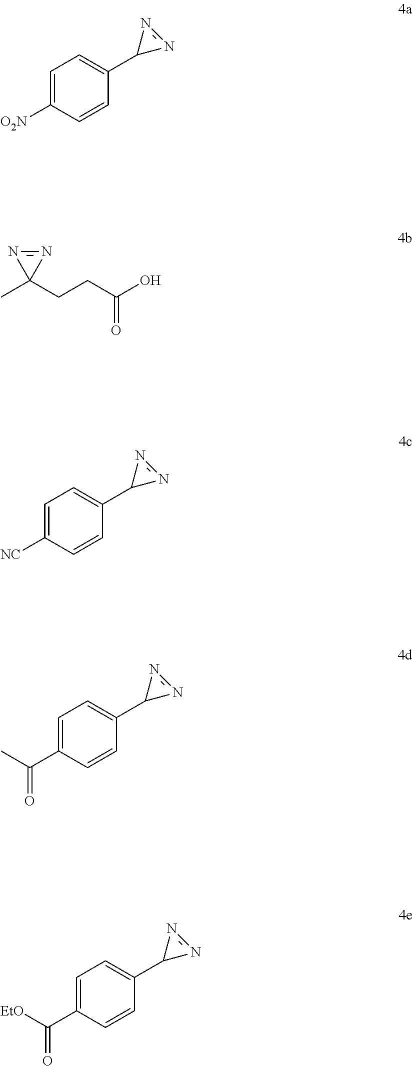

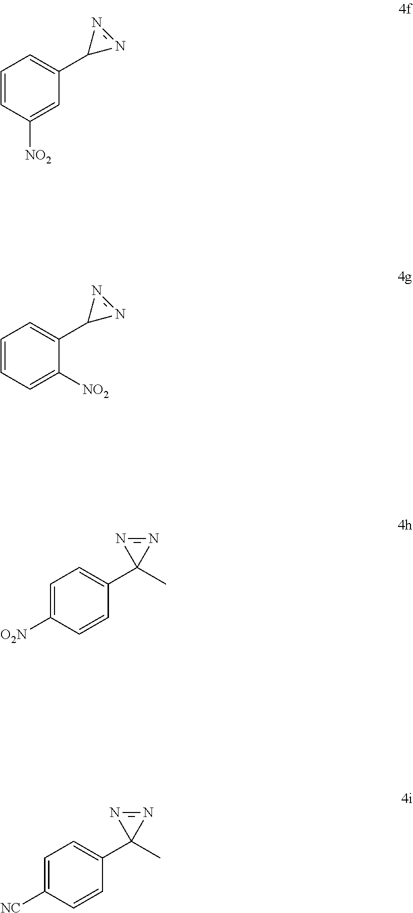

According to another embodiment, the diazirines of formula (I), not enriched in nitrogen-15, obtainable or directly obtained via the method for obtaining diazirines according to the invention, are derived from amino acids, preferably natural amino acids. In particular, the diazirines of formula (I) according to the invention derived from amino acids are capable of being obtained or directly obtained by the method of the invention wherein compound (II) is of formula (IIa). According to this embodiment, the diazirines correspond to the following formula (I-A):

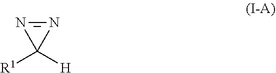

wherein R¹ is as defined above.

The diazirines derived from amino acids advantageously correspond to the following formulas:

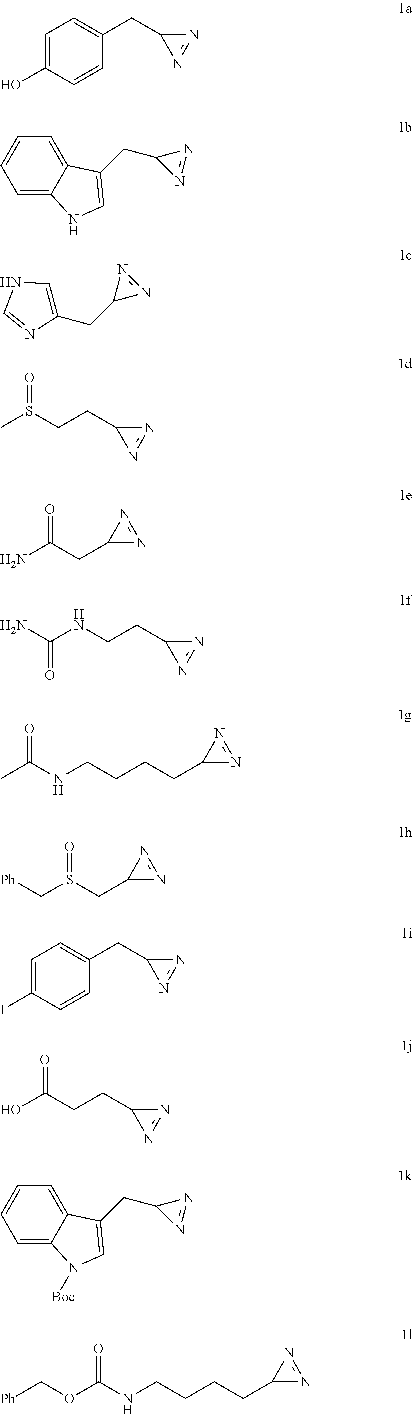

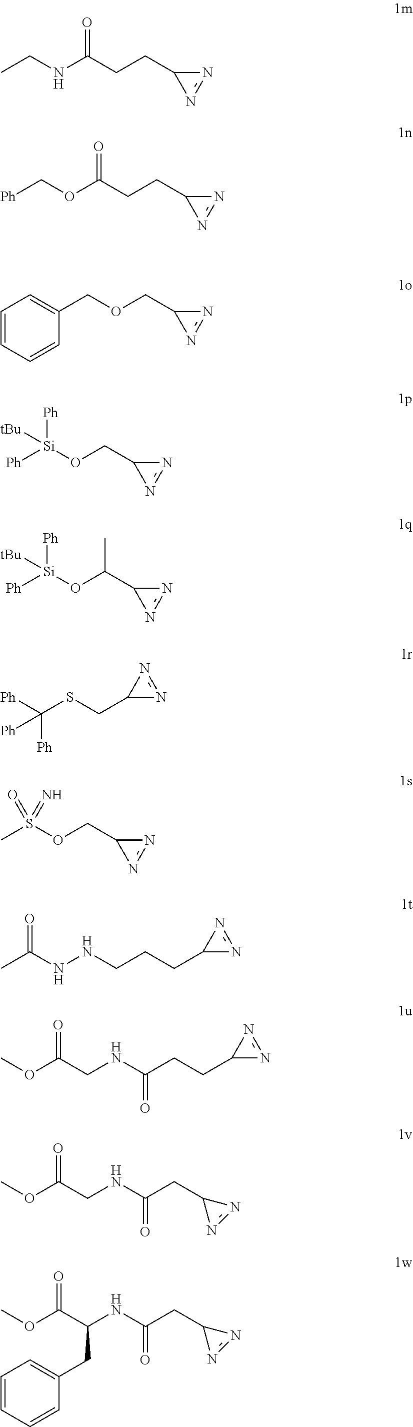

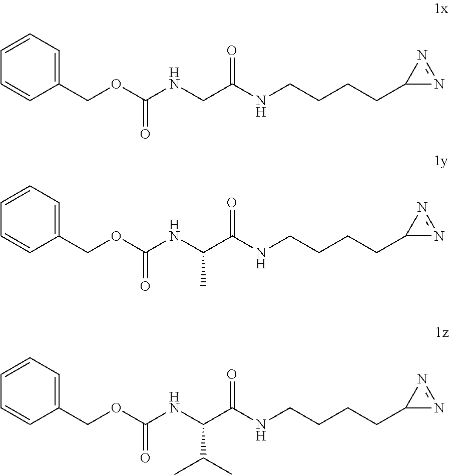 [SMILES: O=C(CNC(=O)OCc1ccccc1)NCCCCC1N=N1 ; C[C@H](NC(=O)OCc1ccccc1)C(=O)NCCCCC1N=N1 ; CC(C)[C@H](NC(=O)OCc1ccccc1)C(=O)NCCCCC1N=N1]

Preferably, they are compounds of the following formulas:

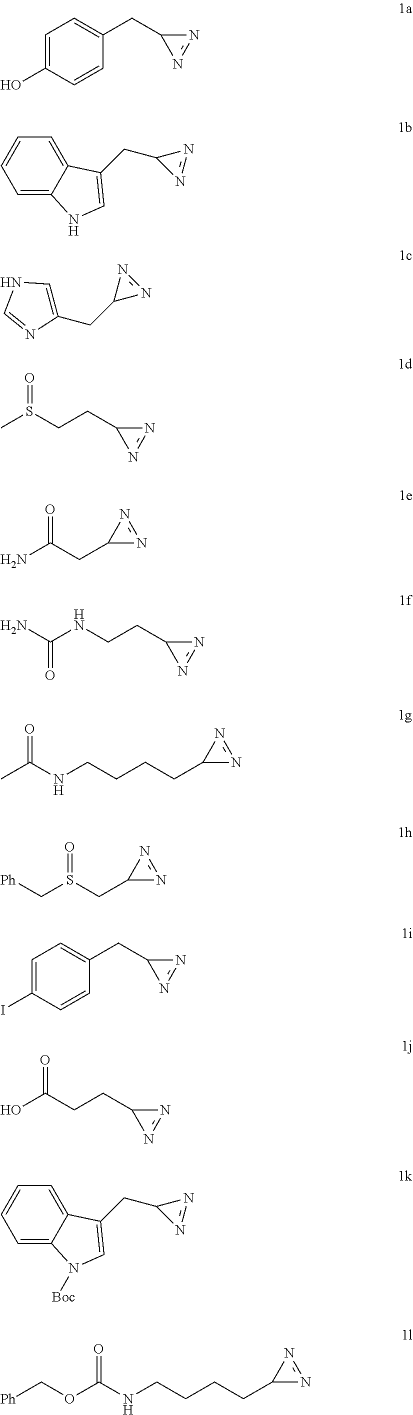

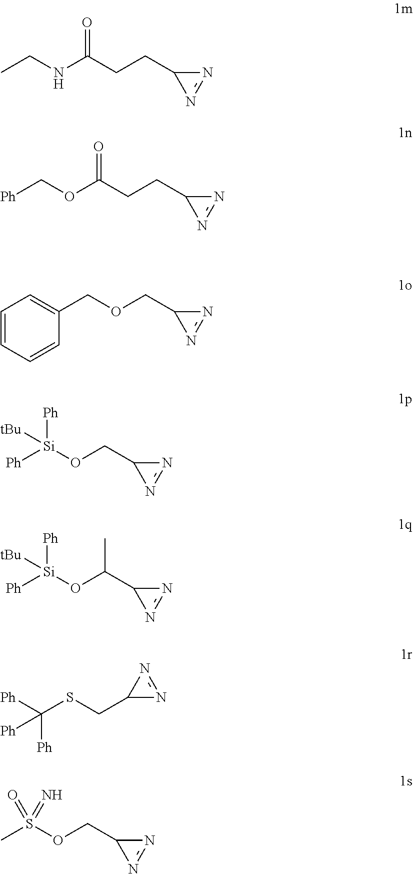

Another subject of the present invention relates to diazirines enriched in nitrogen-15 of the following formula (I′), obtainable or directly obtained by the method for obtaining diazirines according to the present invention:

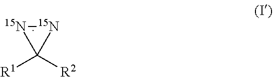

or a physiologically acceptable salt thereof, a stereoisomer or a mixture of stereoisomers in any proportions, wherein:

R¹ represents H, V, W or V—W where:
V represents an aliphatic chain where up to 8, preferably 4, methylene units of the aliphatic chain are optionally replaced by O, C(O), S, S(O), S(O)₂, NR′ or SiR′R″; V is optionally substituted by 1 to 12 groups selected from OH, NR′R″, halogen, CN, oxo, (═NR′) or aryl,
W represents a cycloalkyl, an aryl, a heterocycle or a heteroaryl; W is optionally substituted by 1 to 4 groups selected from halogen, CN, NO₂, OH, NR′R″ an aliphatic chain, where up to 4 methylene units of the aliphatic chain are optionally replaced by O, C(O), S, S(O), S(O)₂, NR′ or SiR′R″,
R′ and R″ each represent, independently of one another, H or an aliphatic chain, where up to 4 methylene units of the aliphatic chain are optionally replaced by O, C(O), S, S(O), S(O)₂, NR′ or SiR′R″, and
R² represents H or an aliphatic chain, where up to 4 methylene units of the aliphatic chain are optionally replaced by O, C(O), S, S(O), S(O)₂, NR′ or SiR′R″.

According to a preferred embodiment, R¹ is the side chain of natural or unnatural, protected or unprotected amino acids.

Advantageously, R² represents a hydrogen or a (C₁-C₆) alkyl group, such as methyl, preferably a hydrogen.

In diazirines of formula (I′), when R² is a methyl group, R¹ is preferably a phenyl substituted by a CN, NO₂, C(O)—(C₁-C₄ alkyl) or C(O)O—(C₁-C₄ alkyl) group. More preferably, R¹ is a phenyl substituted by a nitrile (CN) or NO₂ group.

Diazirines of formula (I) obtainable or directly obtained by the method of the invention advantageously correspond to the following compounds:

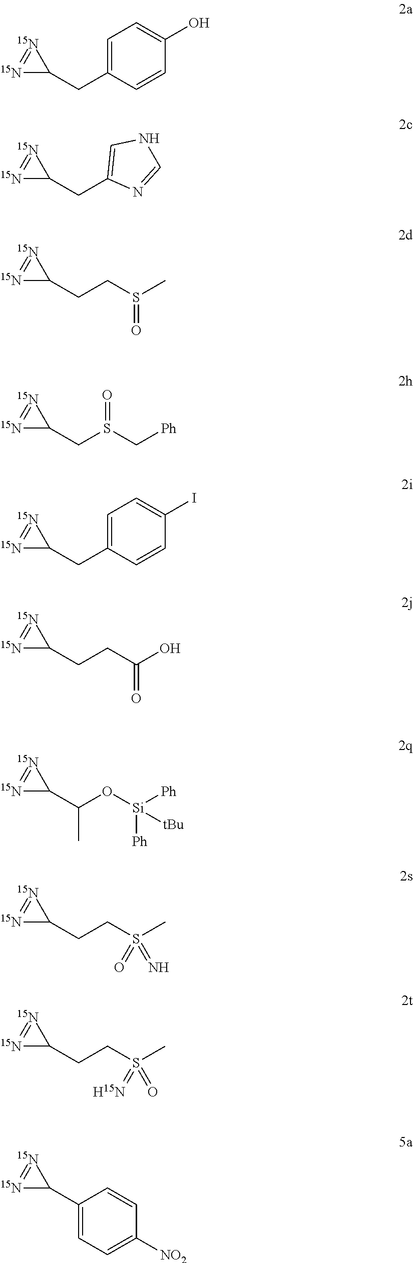

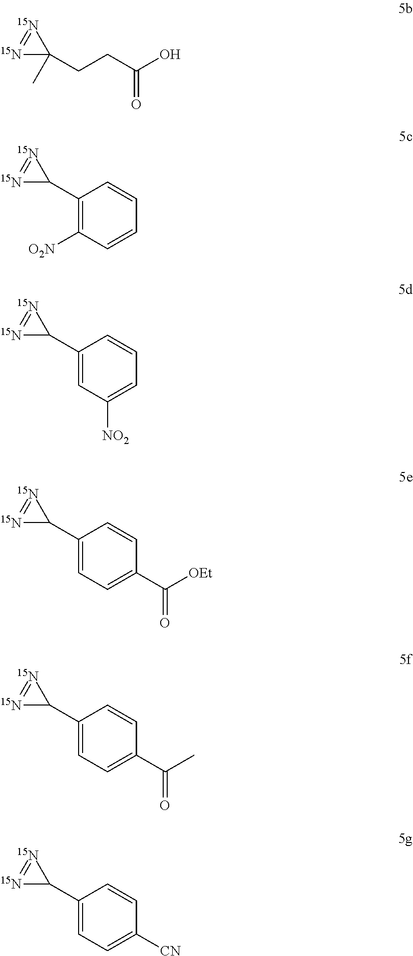

In a particular embodiment, the diazirines of formula (I′) enriched in nitrogen-15 according to the invention are obtainable or directly obtained by the method of the invention wherein compound (II) is of formula (IIb), preferably enriched in nitrogen-15. According to this embodiment, the diazirines of formula (I′) according to the invention advantageously correspond to the following compounds:

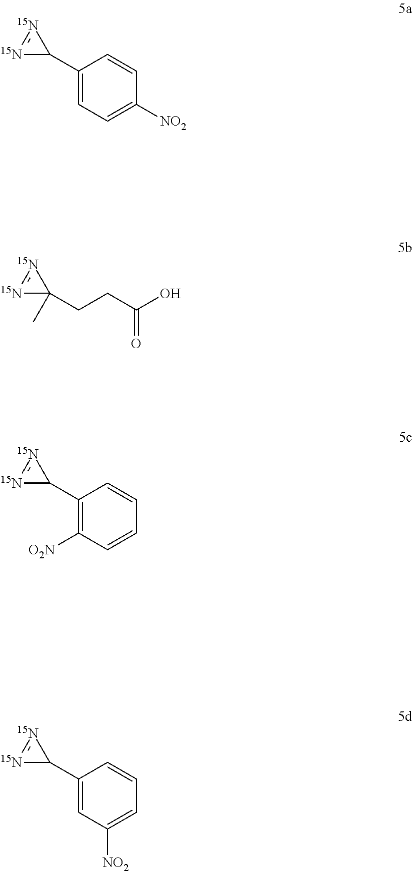

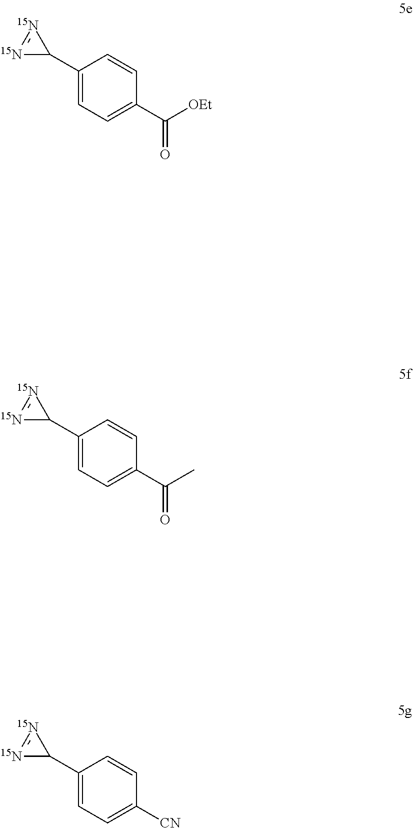

In another embodiment, the diazirines of formula (I′) are obtainable or directly obtained according to the method of the invention from amino acids. These amino acids can be natural or unnatural, preferably natural. Said amino acids can be previously protected as described in the present application. The amino acids may or may not be enriched in nitrogen-15, preferably they are enriched in nitrogen-15. For example, diazirines of formula (I′) result from the following amino acids: L-aspartic acid, L-asparagine, L-glutamine, L-glycine, L-alanine, L-valine, L-isoleucine, L-leucine, L-phenylalanine, L-tryptophan, 4-methyl-L-phenylalanine, L-histidine, L-tyrosine, L-glutamic acid, NE-acetyl-L-lysine, NE-benzyloxycarbamate-L-lysine, L-methionine sulfoxide, S-trityl-L-cysteine, S-benzyl-L-cysteine, O-benzyl-DL-serine, O-tert-butyldiphenylsilyl-DL-serine, O-tert-butyldiphenylsilyl-DL-threonine, 1-(tert-butoxycarbonyl)-L-tryptophan, 4-iodophenyl-alanine, L-theanine, γ-benzyl-L-glutamic acid. In particular, the diazirines of formula (I′) according to the invention derived from amino acids are obtainable or directly obtained by the method of the invention wherein compound (II) is of formula (IIa), preferably enriched in nitrogen-15. According to this embodiment, the diazirines correspond to the following formula (I′-A):

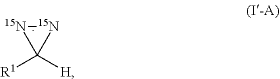

wherein R¹ is as defined above.

Advantageously, the diazirines of formula (I′) derived from amino acids correspond to the following formulas:

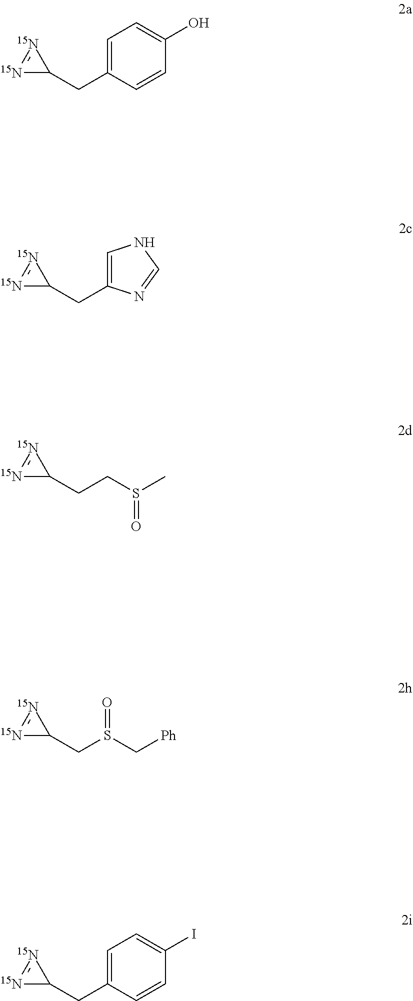

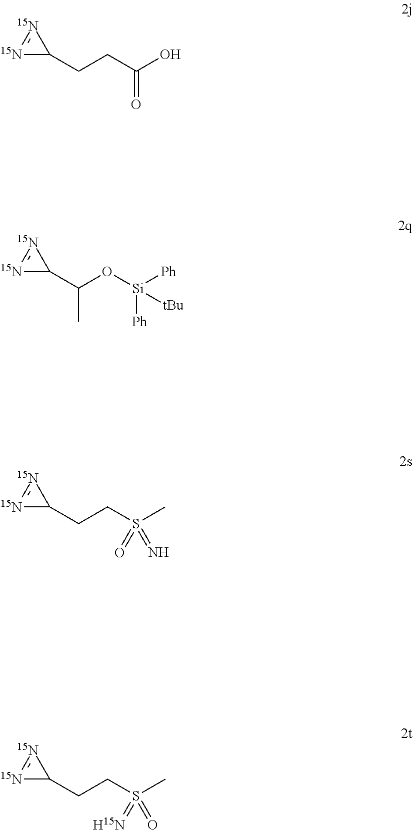

The present invention also concerns the use of diazirines 1a to 1s and optionally diazirines enriched in nitrogen-15 such as defined above for their application in photoaffinity labelling.

The invention also relates to the use of diazirines enriched in nitrogen-15 of formula (I′) for their application in hyperpolarization, notably in the field of medical imaging.

The present application is illustrated by the following examples, without being limited by them.

### EXAMPLES

### General Procedure (A) for the Synthesis of Diazirines 1 from Amino Acids

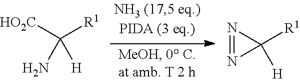

The ammonia solution (1.25 mL at 7 M in methanol; 17.5 eq.) is added into a Schlenk under argon containing the amino acid (0.5 mmol; 1 eq.). The reaction mixture is cooled to 0° C. then the phenyliodonium diacetate obtained (1.5 mmol; 3 eq.) is added in a single portion. After 30 minutes at 0° C., the ice bath is removed, and the reaction mixture is stirred for 1 h 30 min at ambient temperature. After total conversion, the medium is concentrated under reduced pressure then the crude reaction is purified by silica gel chromatography to lead to diazirine 1.

Diazirines synthesized (the amino acid precursor is indicated above the formula):

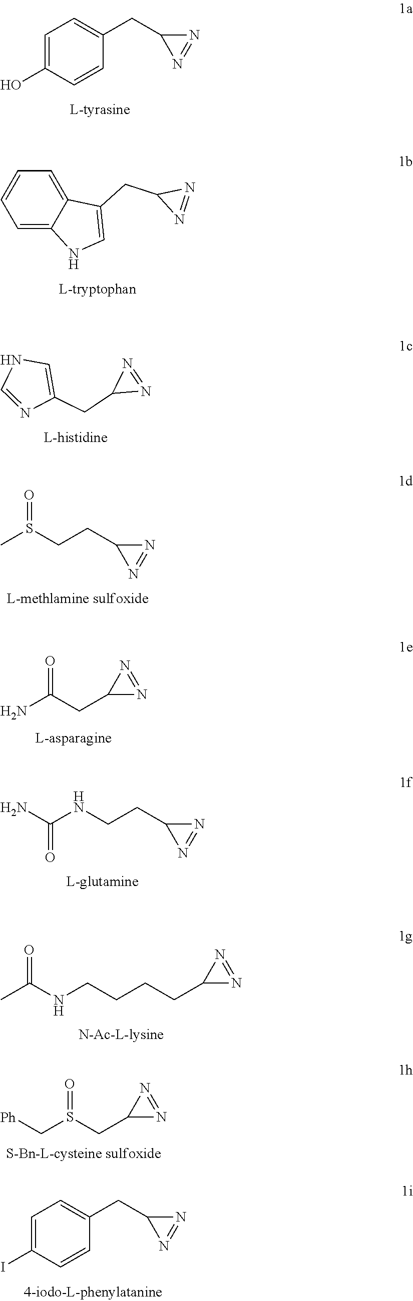

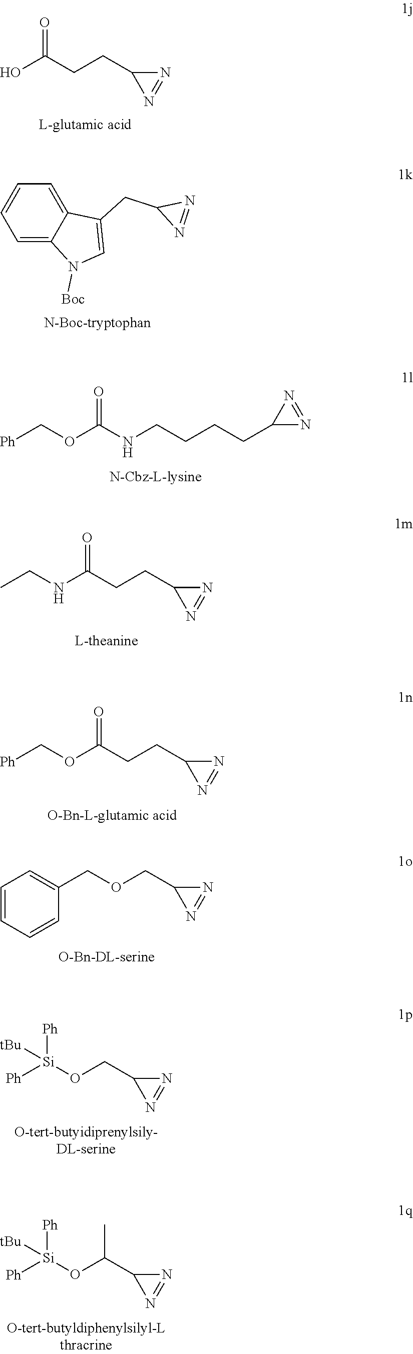

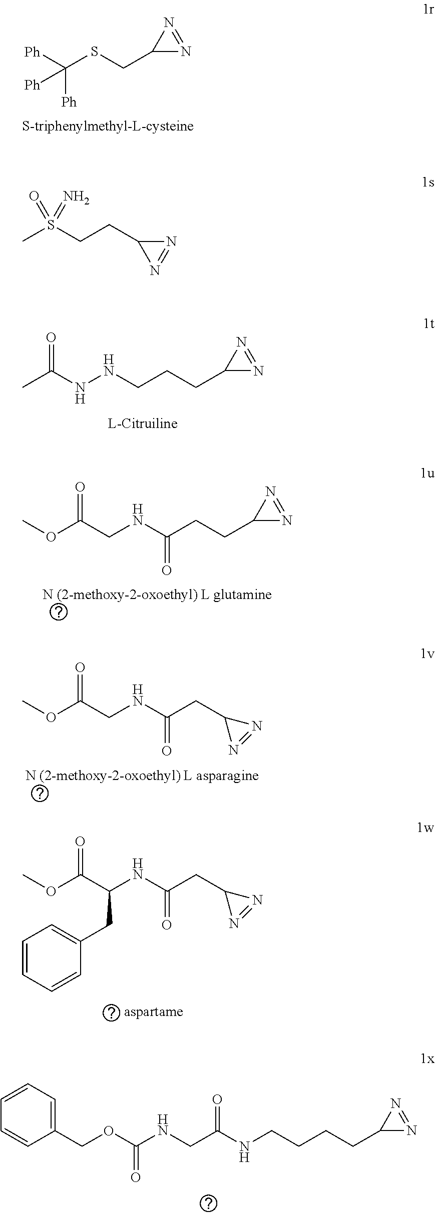

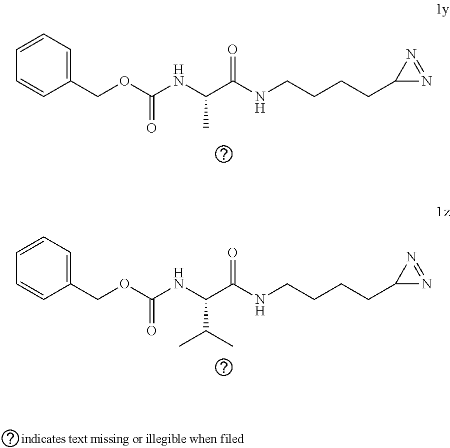 [SMILES: C[C@H](NC(=O)OCc1ccccc1)C(=O)NCCCCC1N=N1 ; CC(C)[C@H](NC(=O)OCc1ccccc1)C(=O)NCCCCC1N=N1]

### 4-((3H-diazirin-3-yl)methyl)phenol (1a)

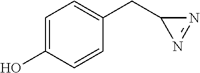 [SMILES: Oc1ccc(CC2N=N2)cc1]

Prepared according to general procedure A by using L-tyrosine (90.6 mg; 0.5 mmol). Potassium hydroxide (56.1 mg; 1 mmol) is added to provide better solubility. After purification by silica gel chromatography (pentane/AcOEt 4/1; R_{f}=0.4), 74 mg of diazirine 1a are obtained (99%) in the form of a brown oil.

¹H NMR (400 MHz, CDCl₃): δ=7.12 (d, J=8.0 Hz, 2H), 6.81 (d, J=8.0 Hz, 2H), 5.03 (broad s, 1H, OH), 2.49 (d, J=4.0 Hz, 2H), 1.10 (t, J=4.0 Hz, 1H).

¹³C NMR (101 MHz, CDCl₃): δ=154.5; 130.2; 128.1; 115.6; 35.7; 22.2.

HRMS (ASAP-QTOF): m/z calculated for C₈H₉N₂O⁺ [M+H]⁺: 149.0715; found: 149.0717.

### 3-((3H-diazirin-3-yl)methyl)-1H-indole (1b)

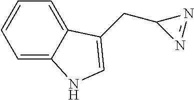 [SMILES: c1ccc2c(CC3N=N3)c[nH]c2c1]

Prepared according to general procedure A by using L-tryptophan (102.1 mg; 0.5 mmol) then purified by silica gel chromatography (pentane/AcOEt 9/1; R_{f}=0.3) to provide 23 mg of diazirine 1b (27%, volatile product) in the form of a brown oil.

¹H NMR (500 MHz, CDCl₃): δ=8.03 (broad s, 1H, NH), 7.68 (d, J=8.0 Hz, 1H), 7.38 (d, J=8.0 Hz, 1H), 7.24 (t, J=8.0 Hz, 1H), 7.20 (t, J=8.0 Hz, 1H), 7.13 (s, 1H), 2.71 (d, J=4.0 Hz, 2H), 1.19 (t, J=4.0 Hz, 1H).

¹³C NMR (125 MHz, CDCl₃): δ=136.4; 127.3; 122.4; 122.3; 119.7; 119.0; 26.5; 21.9.

HRMS (ASAP-QTOF): m/z calculated for C₁₀H₁₀N₃⁺[M+H]⁺: 172.0875; found: 172.0874.

### 5-((3H-diazirin-3-yl)methyl)-1H-imidazole (1c)

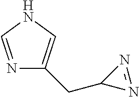

Prepared according to general procedure A by using L-histidine (77.6 mg; 0.5 mmol) then purified by silica gel chromatography (AcOEt/MeOH 95/5; R_{f}=0.3) to provide 60 mg of diazirine 1c (98%) in the form of a light yellow oil.

¹H NMR (500 MHz, CDCl₃): δ=9.48 (broad s, 1H, NH), 7.67 (s, 1H), 6.95 (s, 1H), 2.59 (d, J=4.0 Hz, 2H), 1.17 (t, J=4.0 Hz, 1H).

¹³C NMR (125 MHz, CDCl₃): δ=135.3; 132.9; 116.3; 28.4; 21.0.

HRMS (ESI-QTOF): m/z calculated for C₅H₇N₄⁺[M+H]⁺: 123.0671; found: 123.0673.

### 3-(2-(methylsulfinyl)ethyl)-3H-diazirine (1d)

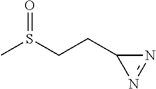

Prepared according to general procedure A by using L-methionine sulfoxide (165.2 mg; 1 mmol) then purified by silica gel chromatography (AcOEt/MeOH 98/2; R_{f}=0.3) to provide 105 mg of diazirine 1d (79%) in the form of a light yellow oil.

¹H NMR (500 MHz, CDCl₃): δ=2.74-2.64 (m, 2H), 2.60 (s, 3H), 1.83-1.68 (m, 2H), 1.06 (t, J=4.5 Hz, 1H).

¹³C NMR (125 MHz, CDCl₃): δ=49.5; 38.5; 23.5; 20.0.

HRMS (ESI-QTOF): m/z calculated for C₄H₈N₂OSNa⁺ [M+Na]⁺: 155.0255; found: 155.0256.

### 2-(3H-diazirin-3-yl)acetamide (1e)

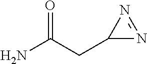

Prepared according to general procedure A by using L-asparagine (66.1 mg; 0.5 mmol) then purified by silica gel chromatography (AcOEt/MeOH 98/2; R_{f}=0.5) to provide 4 mg of diazirine 1e (7%, volatile product) in the form of a light yellow oil.

1H NMR (500 MHz, CDCl₃): δ=5.75 (broad s, 2H), 2.14 (d, J=4.5 Hz, 2H), 1.27 (t, J=4.5 Hz, 1H).

¹³C NMR (125 MHz, CDCl₃): δ=170.7; 37.2; 18.0.

### 3-(3H-diazirin-3-yl)acetamide (1f)

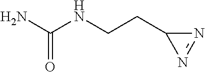

Prepared according to general procedure A by using L-glutamine (73.1 mg; 0.5 mmol) then purified by silica gel chromatography (AcOEt/MeOH 98/2; R_{f}=0.4) to provide 4 mg of diazirine 1f (7%, volatile product) in the form of a light yellow oil.

¹H NMR (500 MHz, DMSO-d6): δ=6.01 (t, J=5.8 Hz, 1H, NH), 5.46 (broad s; 2H, NH₂), 2.98 (td, J=6.9; 5.8 Hz, 2H), 1.27 (td, J=6.9; 4.4 Hz, 2H), 1.10 (t, J=4.4 Hz, 1H).

¹³C NMR (125 MHz, DMSO-d6): δ=158.5, 36.7, 30.6, 19.8.

HRMS (ESI-QTOF): m/z calculated for C₄H₈N₄ONa⁺[M+Na]⁺: 151.0596; found: 151.0597.

### N-(4-(3H-diazirin-3-yl)butyl)acetamide (1 g)

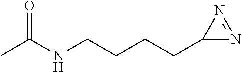

Prepared according to general procedure A by using N_{ε}-acetyl-L-lysine (94.1 mg; 0.5 mmol) then purified by silica gel chromatography (pentane/AcOEt 1/4; R_{f}=0.1) to provide 78 mg of diazirine 1g (99%) in the form of a colorless oil.

¹H NMR (500 MHz, CDCl₃): δ=5.58 (broad s, 1H), 3.23 (q, J=6.5 Hz, 2H), 1.97 (s, 3H), 1.54 (quin, J=7.5 Hz, 2H), 1.36-1.30 (m, 2H), 1.27-1.23 (m, 2H), 0.84 (t, J=4.5 Hz, 1H).

¹³C NMR (125 MHz, CDCl₃): δ=170.1; 39.3; 29.4; 29.1; 23.3; 22.5; 21.2.

HRMS (ESI-QTOF): m/z calculated for C₇H₁₃N₃ONa⁺ [M+Na]⁺: 178.0956; found: 178.0955.

### 3-(1-(benzylsulfinyl)methyl)-3H-diazirine (1h)

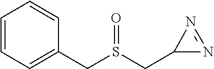 [SMILES: O=S(Cc1ccccc1)CC1N=N1]

Prepared according to general procedure A by using S-benzyl-L-cysteine sulfoxide (113.7 mg; 0.5 mmol) then purified by silica gel chromatography (pentane/AcOEt 1/1; R_{f}=0.2) to provide 69 mg of diazirine 1h (71%) in the form of an orange solid.

¹H NMR (500 MHz, CDCl₃): δ=7.42-7.34 (m, 3H), 7.29-7.27 (m, 2H), 4.20 and 4.18 (AB system, J_{AB}=13.0 Hz, 2H, CH₂), 2.55 (dd, J=14.3 and 5.3 Hz, 1H), 2.40 (dd, J=14.3 and 5.3 Hz, 1H), 1.32 (t, J=5.3 Hz, 1H).

¹³C NMR (125 MHz, CDCl₃): δ=130.2; 129.3; 129.0; 128.8; 58.0; 51.2; 15.7.

### 3-((4-iodophenyl)methyl)-3H-diazirine (1i)

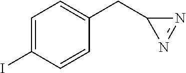 [SMILES: Ic1ccc(CC2N=N2)cc1]

Prepared according to general procedure A by using the trifluoroacetic acid salt of 4-iodo-L-phenylalanine (141.8 mg; 0.35 mmol) then purified by silica gel chromatography (100% pentane; R_{f}=0.6) to provide 70.4 mg of diazirine 1i (78%) in the form of a colorless oil.

¹H NMR (500 MHz, CDCl₃): δ=7.64 (d, J=8.0 Hz, 2H), 7.01 (d, J=8.0 Hz, 2H), 2.49 (d, J=4.3 Hz, 2H), 1.10 (t, J=4.3 Hz, 1H).

¹³C N M R (125 MHz, CDCl₃): δ=138.0; 135.7; 131.0; 92.4; 36.2; 21.6.

HRMS (ASAP-QTOF): m/z calculated for C₈H₈N₂I⁺[M+H]⁺: 258.9732; found: 258.9732.

### 3-(3H-diazirin-3-yl)propanoic acid (1j)

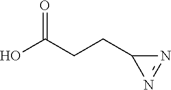

Prepared according to general procedure A by using L-glutamic acid (73.6 mg; 0.5 mmol) then purified by silica gel chromatography (DCM/EtOH 97/3; R_{f}=0.3) to provide 27.6 mg of diazirine 1j (48%) in the form of a light yellow oil.

¹H NMR (500 MHz, CDCl₃): δ=2.41 (t, J=7.3 Hz, 2H), 1.57 (td, J=7.3 and 4.5 Hz, 2H), 0.98 (t, J=4.5 Hz, 1H).

¹³C NMR (125 MHz, CDCl₃): δ=178.7; 29.6; 25.1; 20.6.

HRMS (ESI-QTOF): m/z calculated for C₄H₅N₂O₂⁻ [M]⁻: 113.0351; found: 113.0352.

### 3-((3H-diazirin-3-yl)methyl)-1-(tert-butoxycarbonyl)-indole (1k)

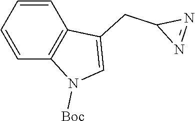 [SMILES: CC(C)(C)OC(=O)n1cc(CC2N=N2)c2ccccc21]

Prepared according to general procedure A by using 1-(tert-butoxycarbonyl)-L-tryptophan (152.2 mg; 0.5 mmol) then purified by silica gel chromatography (pentane/AcOEt 98/2; R_{f}=0.3) to provide 94.3 mg of diazirine 1k (70%) in the form of a colorless oil.

¹H NMR (500 MHz, CDCl₃): δ=8.15 (broad s, 1H), 7.59 (d, J=7.8 Hz, 1H), 7.53 (broad s, 1H), 7.35 (ddd, J=8.3 and 7.2 and 0.9 Hz, 1H), 7.29 (ddd, J=8.0 and 7.1 and 0.9 Hz, 1H), 2.62 (dd, J=4.3 and 0.9 Hz, 2H), 1.19 (t, J=4.3 Hz, 1H). ¹³C NMR (125 MHz, CDCl₃): δ=149.8; 135.7; 130.3; 124.8; 123.8; 122.8; 119.2; 115.5; 115.1; 83.8; 28.3; 26.5; 21.2.

HRMS (ASAP-QTOF): m/z calculated for C₁₅H₁₈N₃O₂⁺ [M+H]⁺: 272.1399; found: 272.1398.

### Benzyl(4-(3H-diazirin-3-yl)butyl carbamate (1l)

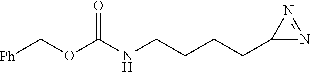 [SMILES: O=C(NCCCCC1N=N1)OCc1ccccc1]

Prepared according to general procedure A by using N_{ε}-benzyloxy carbamate-L-lysine hydrochloric acid (158.4 mg; 0.5 mmol) then purified by silica gel chromatography (pentane/AcOEt 4/1; R_{f}=0.4) to provide 111 mg of diazirine 1l (90%) in the form of a light yellow oil.

¹H NMR (500 MHz, CDCl₃): δ=7.36-7.35 (m, 4H), 7.34-7.30 (m, 1H), 5.10 (s, 2H), 3.20 (q, J=5.5 Hz, 2H), 1.54 (quin, J=6.0 Hz, 2H), 1.37-1.32 (m, 2H), 1.28-1.24 (m, 2H), 0.84 (t, J=3.5 Hz, 1H).

¹³C NMR (125 MHz, CDCl₃): δ=156.5; 136.7; 128.7; 128.3; 66.8; 40.9; 29.6; 29.5; 22.5; 21.4.

HRMS (ESI-QTOF): m/z calculated for C₁₃H₁₇N₃O₂Na⁺[M+Na]⁺270.1218; found: 270.1219.

### 3-(3H-diazirin-3-yl)-N-ethylpropanamide (1m)

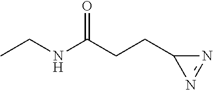

Prepared according to general procedure A by using L-theanine (87 mg; 0.5 mmol) then purified by silica gel chromatography (pentane/AcOEt 30/70; R_{f}=0.3) to provide 34 mg of diazirine 1m (48%) in the form of a colorless oil.

¹H NMR (500 MHz, CDCl₃): δ=5.69 (broad s, 1H), 3.28 (qd, J=7.2 and 5.5 Hz, 2H), 2.14 (t, J=7.4 Hz, 2H), 1.58 (td, J=7.4 and 4.4 Hz, 2H), 1.13 (t, J=7.2 Hz, 3H), 0.95 (t, J=4.4 Hz, 1H).

¹³C NMR (125 MHz, CDCl₃): δ=171.2; 34.6; 31.6; 25.8; 20.9; 14.9.

HRMS (ESI-QTOF): m/z calculated for C₆H₁₁N₃ONa⁺ [M+Na]⁺: 164.0800; found: 164.0801.

### Benzyl-3-(3H-diazirin-3-yl)propanoate (1n)

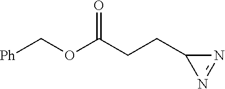 [SMILES: O=C(CCC1N=N1)OCc1ccccc1]

Prepared according to general procedure A by using O-benzyl-L-glutamic acid (118.6 mg; 0.5 mmol) then purified by silica gel chromatography (pentane/AcOEt 4/1; R_{f}=0.7) to provide 100 mg of diazirine 1n (98%) in the form of a light yellow oil.

¹H NMR (500 MHz, CDCl₃): δ=7.39-7.32 (m, 5H), 5.15 (s, 2H), 2.41 (t, J=7.3 Hz, 2H), 1.58 (td, J=7.3 and 4.5 Hz, 2H), 0.96 (t, J=4.5 Hz, 1H).

¹³C NMR (125 MHz, CDCl₃): δ=172.3; 135.8; 128.8; 128.5; 128.4; 66.7; 29.9; 25.4; 20.7.

HRMS (ASAP-QTOF): m/z calculated for C₁₁H₁₃N₂O₂⁺ [M+H]⁺: 205.0977; found: 205.0981.

### 3-((benzyloxy)methyl)-3H-diazirine (10)

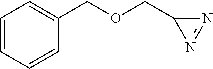 [SMILES: c1ccc(COCC2N=N2)cc1]

Prepared according to general procedure A by using O-benzyl-DL-serine (97.6 mg; 0.5 mmol) then purified by silica gel chromatography (solvent gradient ranging from pentane/AcOEt 3/7 to 100% AcOEt) to provide 43 mg of diazirine 1o (53%) in the form of an orange oil.

¹H NMR (500 MHz, CDCl₃): δ=7.37-7.29 (m, 5H), 4.54 (s, 2H), 3.30 (d, J=4.1 Hz, 2H), 1.13 (t, J=4.1 Hz, 1H).

¹³C NMR (125 MHz, CDCl₃): δ=137.6; 128.6; 128.1; 127.9; 73.0; 69.0; 20.3.

### 3-(((tert-butyldiphenylsilyl)oxy)methyl)-3H-diazirine (1p)

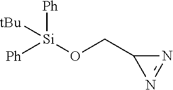 [SMILES: CC(C)(C)[Si](OCC1N=N1)(c1ccccc1)c1ccccc1]

Prepared according to general procedure A by using O-tert-butyldiphenylsilyl-DL-serine (171.7 mg; 0.5 mmol) then purified by silica gel chromatography (solvent gradient ranging from 100% pentane to pentane/AcOEt 10/1) to provide 94 mg of diazirine 1p (60%) in the form of a light yellow oil.

¹H NMR (500 MHz, CDCl₃): δ=7.66-7.64 (m, 4H), 7.46-7.38 (m, 6H), 3.52 (d, J=3.5 Hz, 2H), 1.05 (s broad, 10H)

¹³C NMR (125 MHz, CDCl₃): δ=135.7; 133.1; 130.0; 127.9; 62.7; 26.8; 22.0; 19.4.

### 3-(1-((tert-butyldiphenylsilyl)oxy)ethyl)-3H-diazirine (1q)

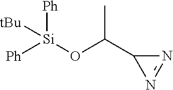 [SMILES: CC(O[Si](c1ccccc1)(c1ccccc1)C(C)(C)C)C1N=N1]

Prepared according to general procedure A by using O-tert-butyldiphenylsilyl-L-threonine (161.0 mg; 0.45 mmol) then purified by silica gel chromatography (solvent gradient ranging from 100% pentane to pentane/Et₂O 100/1) to provide 81.0 mg of diazirine 1q (50%) in the form of a light yellow oil.

¹H NMR (500 MHz, CDCl₃): δ=7.71 (d, J=7.7 Hz, 2H), 7.65 (d, J=7.7 Hz, 2H), 7.45-7.35 (m, 6H), 3.59 (dq, J=6.3 and 3.5 Hz, 1H), 1.07 (s, 9H), 0.97 (d, J=6.4 Hz, 3H), 0.92 (d, J=3.4 Hz, 1H).

¹³C NMR (125 MHz, CDCl₃): δ=136.0; 135.9; 134.0; 133.6; 129.94; 129.88; 127.8; 127.7; 67.7; 27.0; 26.0; 20.4; 19.4.

### 3-(1-(tritylsulfanyl)methyl)-3H-diazirine (1r)

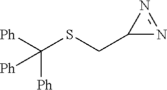 [SMILES: c1ccc(C(SCC2N=N2)(c2ccccc2)c2ccccc2)cc1]

Prepared according to general procedure A by using S-triphenylmethyl-L-cysteine (182 mg; 0.5 mmol) then purified by silica gel chromatography (solvent gradient ranging from 100% pentane to pentane/Et₂O 100/1) to provide 62 mg of diazirine 1r (38%) in the form of a light yellow solid.

¹H NMR (500 MHz, CDCl₃): δ=7.40-7.39 (d, J=7.9 Hz, 6H), 7.29-7.26 (m, 6H), 7.21-7.19 (m, 3H), 2.00 (d, J=4.8 Hz, 2H), 0.65 (t, J=4.8 Hz, 1H).

¹³C NMR (125 MHz, CDCl₃): δ=144.5; 129.7; 128.2; 67.0; 33.6; 20.1.

### 3-(2-(S-methylsulfonimidoyl)ethyl)-3H-diazirine (1s)

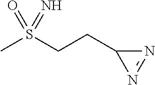

Prepared according to general procedure A with 6 equivalents of PIDA by using L-methionine sulfoxide (82.6 mg; 0.5 mmol) then purified by silica gel chromatography (AcOEt/MeOH 95/5; R_{f}=0.4) to provide 73.6 mg of diazirine 1s (99%) in the form of a brown oil.

¹H NMR (500 MHz, CDCl₃): δ=3.14-3.10 (m, 2H), 3.04 (s, 3H), 1.90 (broad s, NH), 1.90-1.74 (m, 2H), 1.11 (t, J=4.5 Hz, 1H).

¹³C NMR (125 MHz, CDCl₃): δ=52.6; 43.5; 24.2; 19.5.

### N-(3-(3H-diazirin-3-yl)butyl)acetamide (1t)

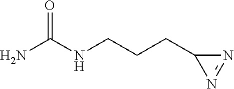

Prepared according to general procedure A by using L-citrulline (87.6; 0.5 mmol) then purified by silica gel chromatography (CH₂Cl₂/MeOH 95/5; R_{f}=0.3) to provide 64.3 mg of diazirine 1t (90%) in the form of a white solid.

Melting point 76.4-79.3° C.

¹H NMR (600 MHz, DMSO-d6): δ=5.93 (t, J=4.8 Hz, 1H, NH), 5.38 (broad s, 2H, NH₂), 2.96 (td, J=6.8; 5.9 Hz, 2H), 1.35-1.30 (m, 2H), 1.18-1.15 (m, 2H), 1.14-1.12 (m, 1H).

¹³C NMR (150 MHz, DMSO-d6): δ=158.7, 38.5, 26.6, 25.7, 20.9.

HRMS (ESI-QTOF): m/z calculated for C₅H₁₀N₄ONa⁺ [M+Na]⁺: 165.0752; found: 165.0752.

### Methyl (3-(3H-diazirin-3-yl)propanoyl)glycinate (1u)

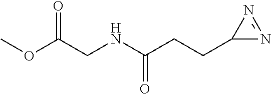

Prepared according to general procedure A by using N_{δ}-(2-methoxy-2-oxoethyl)-L-glutamine (109.1 mg; 0.5 mmol) then purified by silica gel chromatography (pentane/AcOEt 1/1; R_{f}=0.21) to provide 39.7 mg of diazirine 1u (43%) in the form of a yellow oil.

¹H NMR (500 MHz, CDCl₃): δ=6.04 (broad s, 1H, NH), 4.06 (d, J=5.2 Hz, 2H), 3.77 (s, 3H), 2.26 (t, J=7.3 Hz, 1H), 1.61 (dt, J=7.3; 4.5 Hz, 2H), 0.98 (t, J=4.5 Hz, 1H).

¹³C NMR (125 MHz, CDCl₃): δ=171.5, 170.5, 52.6, 41.4, 31.3, 25.7, 20.8.

HRMS (ESI-QTOF): m/z calculated for C₇H₁₁N₃O₃Na⁺[M+Na]⁺: 208.0698; found: 208.0699.

### Methyl (2-(3H-diazirin-3-yl)acetyl)glycinate (1v)

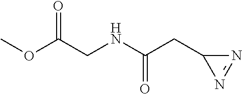

Prepared according to general procedure A by using Nx-(2-methoxy-2-oxoethyl)-L-asparagine (102.1 mg; 0.5 mmol). After purification by silica gel chromatography (pentane/AcOEt 1/1; R_{f}=0.21), 18.4 mg of diazirine 1v are obtained (21%) in the form of a yellow oil.

¹H NMR (500 MHz, CDCl₃): δ=6.19 (broad s, 1H, NH), 4.09 (d, J=5.1 Hz, 2H), 3.78 (s, 3H), 2.16 (d, J=4.7 Hz, 2H), 1.28 (t, J=4.7 Hz, 1H).

¹³C NMR (125 MHz, CDCl₃): δ=170.3, 168.5, 52.7, 41.5, 37.6, 18.1.

HRMS (ESI-QTOF): m/z calculated for C₆H₉N₃O₃Na⁺ [M+Na]⁺: 194.0542; found: 194.0544.

### Methyl (2-(3H-diazirin-3-yl)acetyl)-L-phenylalaninate (1w)

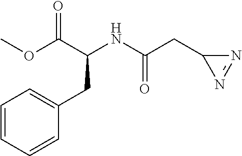 [SMILES: COC(=O)[C@H](Cc1ccccc1)NC(=O)CC1N=N1]

Prepared according to general procedure A by using 6-aspartame (147.2 mg; 0.5 mmol). After purification by silica gel chromatography (pentane/AcOEt 7/3; R_{f}=0.25), 85.8 mg of diazirine 1w are obtained (66%) in the form of a yellow oil.

[α]²⁰_{D}=+58.3° (c 0.5, CHCl₃).

¹H NMR (500 MHz, CDCl₃): δ=7.31-7.24 (m, 3H), 7.13-7.10 (m, 2H), 6.04 (d broad, J=6.6 Hz, 1H, NH), 4.92 (dt, J=7.8; 5.8 Hz, 1H), 4.92 (s, 3H), 3.20 and 3.13 (AB part of an ABX system, J_{AB}=13.9 Hz, J_{AX}=5.8 Hz, 2H, CH₂), 2.13 and 2.03 (AB part of an ABX system, J_{AB}=16.4 Hz, J_{AX}=4.7 Hz, 2H, CH₂), 1.21 (t, J=4.7 Hz, 1H).

¹³C NMR (125 MHz, CDCl₃): δ=171.9, 167.9, 135.6, 129.4, 128.8, 127.4, 53.3, 52.6, 37.9, 37.8, 18.1.

HRMS (ESI-QTOF): m/z calculated for C₁₃H₁₅N₃O₃Na⁺[M+Na]⁺: 284.1011; found: 284.1012.

### Benzyl (2-((4-(3H-diazirin-3-yl)butyl)amino)-2-oxoethyl)carbamate (1x)

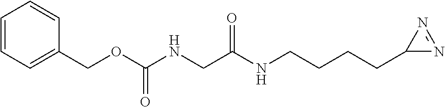 [SMILES: O=C(CNC(=O)OCc1ccccc1)NCCCCC1N=N1]

Prepared according to general procedure A by using Nε-(N-Cbz-glycyl)lysine (168.7 mg; 0.5 mmol). After purification by silica gel chromatography (pentane/AcOEt 2/3; R_{f}=0.33), 107.9 mg of diazirine 1x are obtained (71%) in the form of a yellow solid.

Melting point 80.2-82.2° C.

¹H NMR (600 MHz, CDCl₃): δ=7.38-7.32 (m, 5H), 6.02 (broad s, 1H, NH), 5.39 (broad s, 1H, NH), 5.13 (s, 2H), 3.84 (d broad, J=5.6 Hz, 2H), 3.27-3.24 (m, 2H), 1.56-1.50 (m, 2H), 1.35-1.28 (m, 2H), 1.28-1.23 (m, 2H), 0.83 (t, J=4.0 Hz, 1H).

¹³C NMR (150 MHz, CDCl₃): δ=168.0, 156.8, 136.1, 128.7, 128.5, 128.3, 67.5, 44.9, 39.4, 29.4, 29.1, 22.5, 21.3.

HRMS (ESI-QTOF): m/z calculated for C₁₅H₂₀N₄O₃Na⁺ [M+Na]⁺: 327.1433; found: 327.1435.

### Benzyl (S)-(1-((4-(3H-diazirin-3-yl)butyl)amino)-1-oxopropan-2-yl)carbamate (1y)

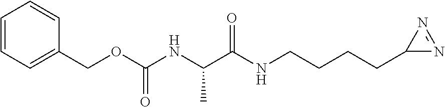 [SMILES: C[C@H](NC(=O)OCc1ccccc1)C(=O)NCCCCC1N=N1]

Prepared according to general procedure A by using Nε-(N-Cbz-alanyl)lysine (175.7 mg; 0.5 mmol). After purification by silica gel chromatography (pentane/AcOEt 1/1; R_{f}=0.25), 144.2 mg of diazirine 1y are obtained (91%) in the form of a beige solid.

[ [symbol] s²⁰_{D}=−20.4° (c 0.5, CHCl₃).

Melting point 78.2-80.3° C.

¹H NMR (500 MHz, CDCl₃): δ=7.37-7.32 (m, 5H), 6.02 (broad s, 1H, NH), 5.23 (broad s, 1H, NH), 5.12 (broad s, 2H), 4.20-4.15 (m, 1H), 3.26-3.22 (m, 2H), 1.55-1.49 (m, 2H), 1.38 (d, J=7.0 Hz, 3H), 1.35-1.28 (m, 2H), 1.27-1.23 (m, 2H), 0.83 (t, J=4.2 Hz, 1H).

¹³C NMR (125 MHz, CDCl₃): δ=172.2, 156.2, 136.2, 128.7, 128.4, 128.3, 67.3, 50.7, 39.4, 29.4, 29.1, 22.5, 21.3, 18.5.

HRMS (ESI-QTOF): m/z calculated for C₁₆H₂₂N₄O₃Na⁺[M+Na]⁺: 341.1590; found: 341.1592.

### Benzyl (S)-(1-((4-(3H-diazirin-3-yl)butyl)amino)-3-methyl-1-oxobutan-2-yl)carbamate (1z)

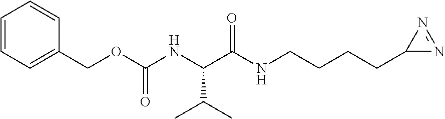 [SMILES: CC(C)[C@H](NC(=O)OCc1ccccc1)C(=O)NCCCCC1N=N1]

Prepared according to general procedure A by using Nε-(N-Cbz-valyl)lysine (189.8 mg; 0.5 mmol). After purification by silica gel chromatography (pentane/AcOEt 7/3; R_{f}=0.25), 70.7 mg of diazirine 1z are obtained (41%) in the form of a beige solid.

[α]²⁰_{D}=−8.7° (c 0.2, CHCl₃).

Melting point 98.3-103.2° C.

¹H NMR (500 MHz, CDCl₃): δ=7.37-7.31 (m, 5H), 5.99 (broad s, 1H, NH), 5.36 (d broad, J=8.2 Hz, 1H, NH), 5.10 (broad s, 2H), 3.92-3.89 (m, 1H), 3.29-3.18 (m, 2H), 2.13-2.08 (m, 1H), 1.56-1.50 (m, 2H), 1.35-1.22 (m, 4H), 0.96 (d, J=6.8 Hz, 1H), 0.92 (d, J=6.8 Hz, 1H), 0.83 (t, J=4.2 Hz, 1H).

¹³C NMR (125 MHz, CDCl₃): δ=171.3, 156.6, 136.3, 128.7, 128.4, 128.2, 67.2, 60.9, 39.3, 31.0, 29.4, 29.1, 22.5, 21.3, 19.4, 18.0.

HRMS (ESI-QTOF): m/z calculated for C₁₈H₂₆N₄O₃Na⁺ [M+Na]⁺: 369.1903; found: 369.1903.

Preparing an ammonia solution labelled with nitrogen-15, at 7 M in methanol (7 M ¹⁵NH₃ in MeOH) Sodium methanolate (3.780 g; 70 mmol) is added to a solution of ammonium chloride enriched in nitrogen-15 (¹⁵NH₄Cl; 3.814 g; 70 mmol) in anhydrous methanol (7-2 mL) at 0° C., in 5 portions over a period of 30 minutes, with vigorous magnetic stirring. The reaction medium is then stirred at ambient temperature for two hours and then put through the centrifuge at 4500 RPM for 10 minutes. The supernatant is then transferred, under a flow of argon, into a sealed bottle.

### General Procedure (B) for the Synthesis of ¹⁵N₂-diazirines 2 from amino acids

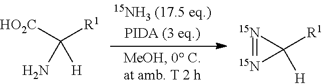

The ammonia solution labelled with nitrogen-15 (1.25 mL at 7 M in methanol; 17.5 eq.) is added into a Schlenk under argon containing amino acid (0.5 mmol; 1 eq.). The reaction mixture is cooled to 0° C. then the phenyliodonium diacetate obtained (1.5 mmol; 3 eq.) is added in a single portion. After 30 minutes at 0° C., the ice bath is removed, and the reaction mixture is stirred for 1 h 30 min at ambient temperature. After total conversion, the medium is concentrated under reduced pressure then the crude reaction is purified by silica gel chromatography to lead to ¹⁵N₂-diazirines 2. ¹⁵N₂-diazirines synthesized (the amino acid precursor is indicated above the formula):

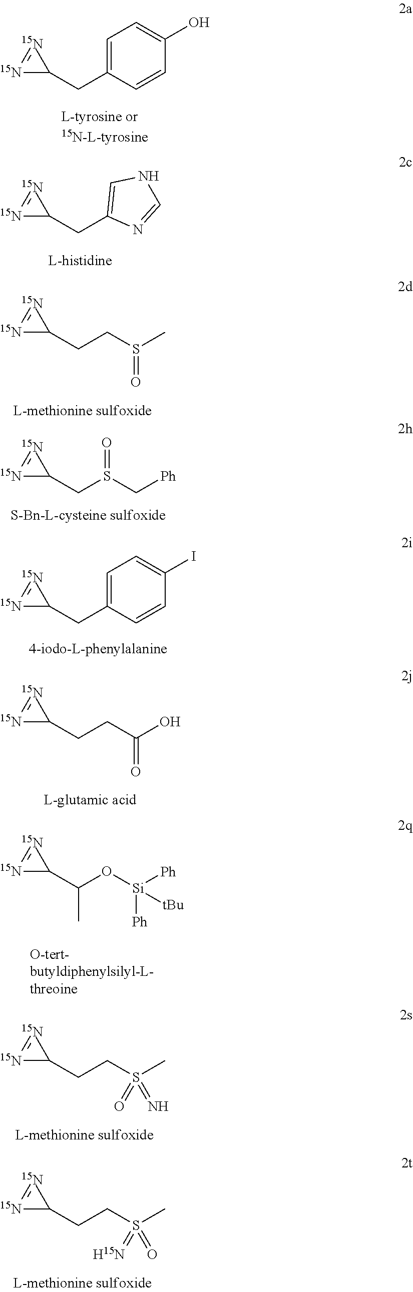

### ¹⁵N₂-(4-((3H-diazirin-3-yl)methyl)phenol) (2a)

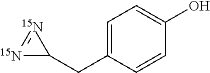 [SMILES: Oc1ccc(CC2[15N]=[15N]2)cc1]

Prepared according to general procedure B by using L-tyrosine (90.6 mg; 0.5 mmol). Potassium hydroxide (56 mg; 1 mmol) is added to provide better solubility. After purification by silica gel chromatography (pentane/AcOEt 4/1; R_{f}=0.4), 68 mg of diazirine 2a (91%) are obtained in the form of a brown oil.

¹H NMR (600 MHz, CDCl₃): δ=7.12 (d, J=8.0 Hz, 2H), 6.80 (d, J=8.0 Hz, 2H), 4.74 (broad s, 1H, OH), 2.49 (d, J=4.3 Hz, 2H), 1.10 (tt, J=4.2 and 2.2 Hz, 1H).

¹³C NMR (150 MHz, CDCl₃): δ=154.6; 130.3; 128.3; 115.7; 35.8; 22.2 (t, J=9.3 Hz).

¹⁵N NMR (60.8 MHz, CDCl₃): δ=445.8 (dt, J=2.2 and 0.8 Hz, 2N).

HRMS (ASAP-QTOF): m/z calculated for C₈H₉¹⁵N₂O⁺[M+H]⁺: 151.0656; found: 151.0658.

¹⁵N₂-diazirine/(¹⁵N-¹⁴N)-diazirine ratio: 87%

By using ¹⁵N-L-tyrosine, the amount of ¹⁵N₂-diazirine is greater than 99%.

### ¹⁵N₂-(5-((3H-diazirin-3-yl)methyl)-1H-imidazole) (2c)

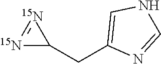

Prepared according to general procedure B by using L-histidine (77.6 mg; 0.5 mmol) then purified by silica gel chromatography (AcOEt/MeOH 95/5; R_{f}=0.3) to provide 35.4 mg of diazirine 2c (57%) in the form of a light yellow oil.

¹H NMR (500 MHz, CDCl₃): δ=11.4 (broad s, 1H, NH), 7.69 (s, 1H), 6.96 (s, 1H), 2.60 (d, J=4.3 Hz, 2H), 1.18 (tt, J=4.2 and 2.5 Hz, 1H).

¹³C NMR (125 MHz, CDCl₃): δ=135.2; 132.8; 116.2; 28.3; 20.9 (t, J=9.3 Hz).

¹⁵N NMR (50.7 MHz, CDCl₃): δ=443.5 (dt, J=2.5 and 0.8 Hz, 2N).

HRMS (ASAP-QTOF): m/z calculated for C₅H₇N₂¹⁵N₂⁺ [M+H]⁺: 125.0611; found: 125.0616.

¹⁵N₂-diazirine/(¹⁵N-¹⁴N)-diazirine ratio: 75%.

### ¹⁵N₂-(3-(2-(methylsulfinyl)ethyl)-3H-diazirine) (2d)

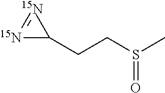

Prepared according to general procedure B by using L-methionine sulfoxide (82.6 mg; 0.5 mmol) then purified by silica gel chromatography (AcOEt/MeOH 98/2; R_{f}=0.3) to provide 54 mg of diazirine 2d (80%) in the form of a light yellow oil.

¹H NMR (500 MHz, CDCl₃): δ=2.74-2.65 (m, 2H), 2.61 (s, 3H), 1.83-1.71 (m, 2H), 1.07 (tt, J=4.2 and 2.2 Hz, 1H).

¹³C NMR (125 MHz, CDCl₃): δ=49.6; 38.7; 23.5; 19.9 (t, J=9.3 Hz).

¹⁵N NMR (50.7 MHz, CDCl₃): δ=442.3 (dt, J=2.5 and 0.8 Hz, 2N).

HRMS (ESI-QTOF): m/z calculated for C₄H₈¹⁵N₂OSNa⁺[M+Na]⁺: 157.0196; found: 157.0197.

¹⁵N₂-diazirine/(¹⁵N-¹⁴N)-diazirine ratio: 75%.

### ¹⁵N₂-(3-(1-(benzylsulfinyl)methyl)-3H-diazirine) (2h)

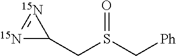 [SMILES: O=S(Cc1ccccc1)CC1[15N]=[15N]1]

Prepared according to general procedure B by using S-benzyl-L-cysteine sulfoxide (113.7 mg; 0.5 mmol) then purified by silica gel chromatography (pentane/AcOEt 1/1; R_{f}=0.3) to provide 49 mg of diazirine 2h (50%) in the form of an orange solid.

¹H NMR (500 MHz, CDCl₃): δ=7.42-7.34 (m, 3H), 7.29-7.27 (m, 2H), 4.19 and 4.16 (AB system, J_{AB}=13.1 Hz, 2H, CH₂), 2.53 (dd, J=14.2 and 4.8 Hz, 1H), 2.40 (dd, J=14.2 and 4.8 Hz, 1H), 1.32 (tt, J=4.9 and 2.2 Hz, 1H).

¹³C NMR (125 MHz, CDCl₃): δ=130.0; 129.2; 128.9; 128.7; 57.9; 51.2; 15.5 (t, J=9.3 Hz).

¹⁵N NMR (60.8 MHz, CDCl₃): δ=437.2 (ddt, J=13.7 and 2.2 and 0.8 Hz), 436.2 (ddt, J=16.7 and 2.2 and 0.8 Hz).

HRMS (ESI-QTOF): m/z calculated for C₉H₁₁¹⁵N₂OS⁺[M+H]⁺: 197.0533; found: 197.0538.

¹⁵N₂-diazirine/(¹⁵N-¹⁴N)-diazirine ratio: 60%.

### ¹⁵N₂-(3-(4-iodobenzyl)-3H-diazirine) (2i)

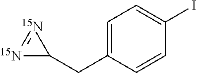 [SMILES: Ic1ccc(CC2[15N]=[15N]2)cc1]

Prepared according to general procedure B by using the trifluoroacetic acid salt of 4-iodo-L-phenylalanine (202.6 mg; 0.5 mmol) then purified by silica gel chromatography (100% pentane; R_{f}=0.6) to provide 97.4 mg of diazirine 7 (75%) in the form of a colorless oil.

¹H NMR (500 MHz, CDCl₃): δ=7.65 (d, J=8.0 Hz, 2H), 7.01 (d, J=8.0 Hz, 2H), 2.49 (d, J=4.6 Hz, 2H), 1.11-1.08 (m, 1H).

¹³C NMR (125 MHz, CDCl₃): δ=137.9; 135.7; 131.0; 92.4; 36.2; 21.6 (t, J=9.4 Hz).

¹⁵N NMR (50.7 MHz, CDCl₃): δ=444.4 (dt, J=2.5 and 0.8 Hz, 2N).

### ¹⁵N₂-(3-(3H-diazirin-3-yl)propanoic acid) (2j)

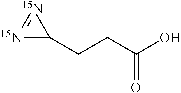

Prepared according to general procedure B by using L-glutamic acid (73.6 mg, 0.5 mmol) then purified by silica gel chromatography (DCM/EtOH 97/3; R_{f}=0.3) to provide 28 mg of diazirine 2j (48%) in the form of a light yellow oil.

¹H NMR (500 MHz, CDCl₃): δ=2.42 (t, J=7.2 Hz, 2H), 1.59-1.55 (m, 2H), 0.99-0.96 (m, 1H).

¹³C NMR (125 MHz, CDCl₃): δ=178.6; 29.6; 25.1; 20.5 (t, J=9.2 Hz).

¹⁵N NMR (50.7 MHz, CDCl₃): δ=443.8 (dt, J=2.5 and 0.8 Hz, 2N).

### ¹⁵N₂-(3-(1-((tert-butyldiphenylsilyl)oxy)ethyl)-3H-diazirine) (2q)

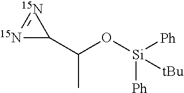 [SMILES: CC(O[Si](c1ccccc1)(c1ccccc1)C(C)(C)C)C1[15N]=[15N]1]

Prepared according to general procedure B by using O-tert-butyldiphenylsilyl-L-threonine (178.8 mg; 0.5 mmol) then purified by silica gel chromatography (solvent gradient ranging from 100% pentane to pentane/Et₂O 100/1) to provide 77 mg of diazirine 2q (47%) in the form of a light yellow oil.

¹H NMR (500 MHz, CDCl₃): δ=7.71 (d, J=7.4 Hz, 2H), 7.65 (d, J=7.4 Hz, 2H), 7.46-7.36 (m, 6H), 3.59 (m, 1H), 1.07 (s, 9H), 0.97 (d, J=6.2 Hz, 3H), 0.93-0.91 (m, 1H).

¹³C NMR (125 MHz, CDCl₃): δ=136.0; 135.9; 134.0; 133.6; 129.95; 129.89; 127.82; 127.75; 67.7; 27.0; 25.9 (dd, J=9.4 and 7.3 Hz); 20.4; 19.4.

¹⁵N NMR (50.7 MHz, CDCl₃): δ=438.3 (ddt, J=13.9 and 2.9 and 0.8 Hz), 439.8 (ddt, J=16.8 and 2.9 and 0.8 Hz).

### ¹⁵N₂-(3-(2-(S-methylsulfonimidoyl)ethyl)-3H-diazirine) (2s)

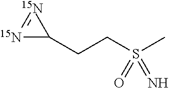

Ammonium carbamate (47 mg; 0.6 mmol; 4 eq.) then phenyliodonium diacetate (144 mg; 0.45 mmol; 3 eq.) are added in a single portion into a sample holder containing diazirine 2d (20 mg; 0.15 mmol) in solution in methanol (0.3 mL) at ambient temperature. The reaction mixture is stirred for 30 minutes at ambient temperature. After total conversion, the medium is concentrated under reduced pressure then the crude reaction is purified by silica gel chromatography (AcOEt/MeOH 95/5; R_{f}=0.4) to provide 19 mg of diazirine sulfoximine 2s (85%) in the form of a brown oil.

¹H NMR (500 MHz, CDCl₃): δ=3.10 (t, J=7.7 Hz, 2H), 3.02 (s, 3H), 2.05 (broad s, NH), 1.88-1.76 (m, 2H), 1.09 (tt, J=4.4 and 2.2 Hz, 1H).

¹³C NMR (125 MHz, CDCl₃): δ=52.7; 43.6; 24.3; 19.6 (t, J=9.3 Hz).

¹⁵N NMR (50.7 MHz, CDCl₃): δ=441.7 (m, 2N).

### ¹⁵N₂-(3-(2-(S-methyl-¹⁵N-sulfonimidoyl)ethyl)-3H-diazirine) (2t)

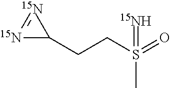

Prepared according to general procedure B with 6 equivalents of PIDA by using L-methionine sulfoxide (82.6 mg; 0.5 mmol) then purified by silica gel chromatography (AcOEt/MeOH 95/5; R_{f}=0.4) to provide 75.1 mg of diazirine 2t (99%) in the form of a brown oil.

¹H NMR (500 MHz, CDCl₃): δ=3.10 (t, J=7.7 Hz, 2H), 3.02 (s, 3H), 2.05 (broad s, NH), 1.88-1.76 (m, 2H), 1.09 (tt, J=4.4 and 2.2 Hz, 1H).

¹³C NMR (125 MHz, CDCl₃): δ=52.7 (d, J=3.1 Hz, 1C); 43.6 (d, J=4.3 Hz, 1C); 24.3; 19.6 (t, J=9.3 Hz, 1C).

¹⁵N NMR (50.7 MHz, CDCl₃): δ=441.7 (m, 2N), 92.4 (m, 1N).

### General Procedure (C) for Synthesis of N-tert-butyl³ imines 3a to 3g

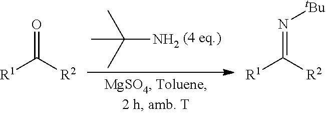

The carbonyl derivative is added portion- or dropwise (1 eq.) to a suspension of tert-butyl amine (4 eq.) in anhydrous toluene (1.1 mL) containing 500 mg of anhydrous magnesium sulfate, over a period of 20 minutes. The solution is stirred for 2 hours at ambient temperature. After total conversion, the medium is filtered through a frit and the residue is washed with chloroform, then the solvent is evaporated off under reduced pressure to provide one of imines 3a to 3g.

### N-tert-butyl-1-(4-nitrophenyl)methanimine (3a)

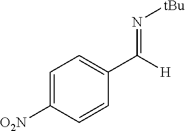 [SMILES: CC(C)(C)/N=C/c1ccc([N+](=O)[O-])cc1]

Prepared according to general procedure C by using p-nitrobenzaldehyde (1000 mg; 6.62 mmol) to provide 1.305 g of imine 3a (96%) in the form of a yellow solid.

¹H NMR (500 MHz, CDCl₃): δ=8.33 (s, 1H), 8.26 (d, J=8.5 Hz, 2H), 7.92 (d, J=8.5 Hz, 2H), 1.32 (s, 9H).

¹³C NMR (125 MHz, CDCl₃): δ=153.1; 130.6; 128.8; 124.5; 123.9; 29.6; 27.8.

HRMS (ASAP-QTOF): m/z calculated for C₁₁H₁₅N₂O₂⁺ [M+H]⁺: 207.1134; found: 207.1133.

### 4-(tert-butylimino)pentanoic acid (3b)

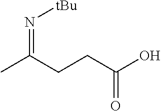

Prepared according to general procedure C by using 4-oxopentanoic acid (1000 mg; 8.61 mmol) to provide 1.474 g of imine 3b (99%) in the form of a beige powder.

¹H NMR (500 MHz, CDCl₃): δ=7.55 (broad s, 1H, OH), 2.67 (t, J=6.8 Hz, 2H), 2.40 (t, J=6.8 Hz, 2H), 2.14 (s, 3H), 1.30 (s, 9H).

¹³C NMR (125 MHz, CDCl₃): δ=208.8; 179.0; 50.7; 40.0; 32.1; 30.2; 28.1.

HRMS (ESI-QTOF): m/z calculated for C₉H₁₇NO₂Na⁺[M+Na]⁺: 194.1157; found: 194.1155.

### 4-((tert-butylimino)methyl)benzonitrile (3c)

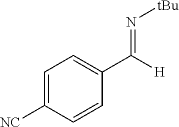 [SMILES: CC(C)(C)/N=C/c1ccc(C#N)cc1]

Prepared according to general procedure C by using 4-formylbenzonitrile (1000 mg; 7.63 mmol) to provide 1.422 g of imine 3c (94%) in the form of a yellow solid.

Melting point 64.3-66.9° C.

¹H NMR (500 MHz, CDCl₃): δ=8.27 (s 1H), 7.85 (d, J=8.2 Hz, 2H), 7.69 (d, J=8.2 Hz, 2H), 1.30 (s, 9H).

¹³C NMR (125 MHz, CDCl₃): δ=153.4, 141.1, 132.5, 128.5, 118.9, 113.5, 58.2, 29.7.

HRMS (ESI-QTOF): m/z calculated for C₁₂H₁₅N₂⁺ [M+H]⁺: 187.1235; found: 187.1234.

### ethyl 4-((tert-butylimino)methyl)benzoate (3d)

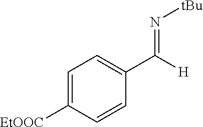 [SMILES: CCOC(=O)c1ccc(/C=N/C(C)(C)C)cc1]

Prepared according to general procedure C by using ethyl 4-formylbenzoate (500 mg; 2.8 mmol) to provide 351 mg of imine 3d (54%) in the form of a yellow solid.

¹H NMR (500 MHz, CDCl₃): δ=8.66 (s, 1H), 8.07 (d, J=8.3 Hz, 2H), 7.81 (d, J=8.3 Hz, 2H), 4.39 (q, J=7.1 Hz, 1H), 1.41 (t, J=7.1 Hz, 3H), 1.31 (s, 9H).

¹³C NMR (125 MHz, CDCl₃): δ=166.5, 154.5, 141.1, 131.9, 129.9, 127.9, 61.3, 57.9, 29.8, 14.5.

HRMS (ESI-QTOF): m/z calculated for C₁₄H₂₀NO₂+[M+H]⁺: 234.1494; found: 234.1493.

### 1-(4-((tert-butylimino)methyl)phenyl)ethan-1-one (3e)

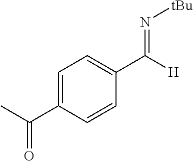

Prepared according to general procedure C by using 4-acetylbenzaldehyde (500 mg; 3.37 mmol) to provide 688.5 mg of imine 3e (>99%) in the form of a yellow solid.

¹H NMR (600 MHz, CDCl₃): δ=8.31 (s, 1H), 7.99 (d, J=8.4 Hz, 2H), 7.84 (d, J=8.4 Hz, 2H), 2.67 (s, 3H), 1.31 (s, 9H).

¹³C NMR (150 MHz, CDCl₃): δ=198.0, 154.3, 141.3, 138.3, 129.7, 128.2, 58.0, 29.8, 27.0.

### N-tert-butyl-1-(3-nitrophenyl)methanimine (3f)

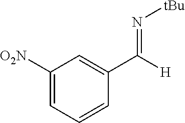 [SMILES: CC(C)(C)/N=C/c1cccc([N+](=O)[O-])c1]

Prepared according to general procedure C by using 3-nitrobenzaldehyde (500 mg; 3.31 mmol) to provide 534 mg of imine 3f (78%) in the form of a beige solid.

Melting point 70.2-73.6° C.

¹H NMR (600 MHz, CDCl₃): δ=8.59-8.58 (m, 1H), 8.32 (s 1H), 8.25-8.23 (m, 1H), 8.10-8.09 (m, 1H), 7.58 (t, J=7.9 Hz, 1H), 1.31 (s, 9H).

¹³C NMR (150 MHz, CDCl₃): δ=152.7, 148.7, 139.0, 133.6, 129.6, 124.7, 122.9, 58.1, 29.7.

HRMS (ESI-QTOF): m/z calculated for C₁₁H₁₅N₂O₂⁺ [M+H]⁺: 207.1134; found: 207.1133.

### N-tert-butyl-1-(2-nitrophenyl)methanimine (3g)

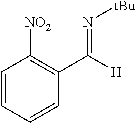 [SMILES: CC(C)(C)/N=C/c1ccccc1[N+](=O)[O-]]

Prepared according to general procedure C by using 2-nitrobenzaldehyde (1000 mg; 6.62 mmol) to provide 1185 mg of imine 3g (87%) in the form of a yellow solid.

¹H NMR (500 MHz, CDCl₃): δ=8.31 (s, 1H), 8.03-7.99 (m, 2H), 7.67-7.64 (m, 1H), 7.55-7.52 (m, 1H), 1.33 (s, 9H).

¹³C NMR (125 MHz, CDCl₃): δ=152.1, 148.9, 133.7, 132.7, 130.3, 129.8, 124.4, 58.5, 29.6.

HRMS (ESI-QTOF): m/z calculated for C₁₁H₁₅N₂O₂⁺ [M+H]⁺: 207.1134; found: 207.1131.

### General Procedure (D) for Synthesis of N-tosyl imines 3h to 3m

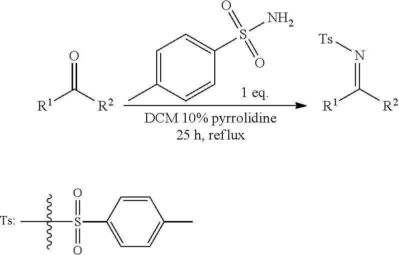

The carbonyl derivative (1 eq.) is added in one portion to a suspension of p-toluenesulfonamide (1 eq.) in anhydrous dichloromethane (0.3 M) containing 10 mol % of pyrrolidine and the 4 Å molecular sieve (1 g/mmol). The solution is stirred for 25 hours at reflux. After total conversion, the medium is filtered through celite and the residue is washed with dichloromethane, then the solvent is evaporated under reduced pressure to provide one of imines 3h to 3m.

### 4-methyl-N-(4-nitrobenzylidene)benzenesulfonamide (3h)

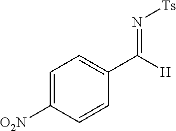 [SMILES: Cc1ccc(S(=O)(=O)/N=C/c2ccc([N+](=O)[O-])cc2)cc1]

Prepared according to general procedure D by using 4-nitrobenzaldehyde (1000 mg; 6.62 mmol) to provide 1576 mg of imine 3h (78%) in the form of a yellow solid.

Melting point 193.4-195.1° C.

¹H NMR (600 MHz, CDCl₃): δ=9.10 (s, 1H), 8.33 (d, J=8.4 Hz, 2H), 8.11 (d, J=8.4 Hz, 2H), 7.90 (d, J=8.0 Hz, 2H), 7.38 (d, J=8.0 Hz, 2H), 2.46 (s, 3H).

¹³C NMR (150 MHz, CDCl₃): δ=167.3, 151.2, 145.4, 137.4, 134.1, 131.9, 130.0, 129.8, 128.4, 126.5, 124.2, 21.8.

HRMS (ESI-QTOF): m/z calculated for C₁₄H₁₃N₂O₄S⁺[M+H]⁺: 305.0596; found: 305.0593.

### N-(4-cyanobenzylidene)-4-methylbenzenesulfonamide (3i)

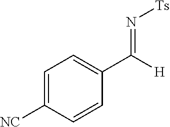 [SMILES: Cc1ccc(S(=O)(=O)/N=C/c2ccc(C#N)cc2)cc1]

Prepared according to general procedure D by using 4-formylbenzonitrile (500 mg; 3.81 mmol) to provide 947 mg of imine 3i (87%) in the form of a beige solid.

Melting point 174.0-176.0° C.

¹H NMR (500 MHz, CDCl₃): δ=9.05 (s, 1H), 8.03 (d, J=8.3 Hz, 2H), 7.89 (d, J=7.9 Hz, 2H), 7.77 (d, J=8.3 Hz, 2H), 7.37 (d, J=7.9 Hz, 2H), 2.45 (s, 3H).

¹³C NMR (125 MHz, CDCl₃): δ=167.9, 145.4, 136.1, 134.4, 132.9, 131.4, 130.1, 128.5, 117.8, 117.7, 21.9.

HRMS (ESI-QTOF): m/z calculated for C₁₅H₁₃N₂O₂S⁺[M+H]⁺: 285.0698; found: 285.0697.

### ethyl 4-((tosylimino)methyl)benzoate (3j)

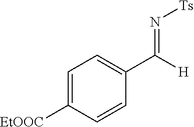 [SMILES: CCOC(=O)c1ccc(/C=N/S(=O)(=O)c2ccc(C)cc2)cc1]

Prepared according to general procedure D by using ethyl 4-formylbenzoate (500 mg; 2.8 mmol) to provide 839.4 mg of imine 3j (90%) in the form of a white solid.

Melting point 154.8-157.5° C.

¹H NMR (600 MHz, CDCl₃): δ=9.06 (s, 1H), 8.14 (d, J=8.5 Hz, 2H), 7.99 (d, J=8.5 Hz, 2H), 7.90 (d, J=8.1 Hz, 2H), 7.37 (d, J=8.1 Hz, 2H), 4.41 (q, J=7.1 Hz, 2H), 2.45 (s, 3H), 1.41 (t, J=7.1 Hz, 3H).

¹³C NMR (150 MHz, CDCl₃): δ=169.1, 165.6, 145.1, 135.9, 135.8, 134.7, 131.1, 130.2, 130.1, 128.4, 61.8, 21.9, 14.4.

HRMS (ESI-QTOF): m/z calculated for C₁₇H₁₈NO₄S⁺[M+H]⁺: 332.0957; found: 332.0959.

### N-(4-acetylbenzylidene)-4-methylbenzenesulfonamide (3k)

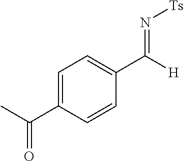 [SMILES: CC(=O)c1ccc(/C=N/S(=O)(=O)c2ccc(C)cc2)cc1]

Prepared according to general procedure by using 4-acetyenzalehyde (500 mg; 3.37 mmol) to provide 1001.4 mg of imine 3k (99%) in the form of an orange solid.

Melting point 156.3-159.9° C.

¹H NMR (500 MHz, CDCl₃): δ=9.00 (s, 1H), 7.98-7.94 (m, 4H), 7.84 (d, J=8.4 Hz, 2H), 7.30 (d, J=8.4 Hz, 2H), 2.58 (s, 3H), 2.38 (s, 3H).

¹³C NMR (125 MHz, CDCl₃): δ=197.3, 168.9, 145.2, 141.5, 136.0, 134.7, 131.5, 130.1, 128.9, 128.4, 27.1, 21.9.

### 4-methyl-N-(3-nitrobenzylidene)benzenesulfonamide (3L)

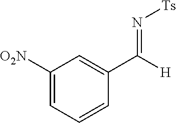 [SMILES: Cc1ccc(S(=O)(=O)/N=C/c2cccc([N+](=O)[O-])c2)cc1]

Prepared according to general procedure D by using 3-nitrobenzaldehyde (500 mg; 3.31 mmol) to provide 878 mg of imine 31(87%) in the form of a beige solid.

Melting point 137.8-140.2° C.

¹H NMR (600 MHz, CDCl₃): δ=9.10 (s, 1H), 8.77 (s, 1H), 8.45 (m, 1H), 8.25 (d, J=7.2 Hz, 2H), 7.91 (d, J=7.2 Hz, 2H), 7.72 (t, J=7.8 Hz, 2H), 7.38 (d, J=7.2 Hz, 2H), 2.46 (s, 3H).

¹³C NMR (150 MHz, CDCl₃): δ=167.4, 145.5, 136.5, 134.3, 134.1, 130.5, 130.2, 129.9, 128.8, 128.5, 125.6, 21.9.

HRMS (ESI-QTOF): m/z calculated for C₁₄H₁₃N₂O₄S⁺[M+H]⁺: 305.0596; found: 305.0596.

### 4-methyl-N-(2-nitrobenzylidene)benzenesulfonamide (3m)

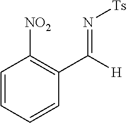 [SMILES: Cc1ccc(S(=O)(=O)/N=C/c2ccccc2[N+](=O)[O-])cc1]

Prepared according to general procedure D by using 2-nitrobenzaldehyde (500 mg; 3.31 mmol) to provide 999 mg of imine 3m (99%) in the form of a yellow solid.

Melting point 108.8-111.0° C.

¹H NMR (600 MHz, CDCl₃): δ=9.46 (s, 1H), 8.16-8.11 (m, 2H), 7.92 (d, J=8.3 Hz, 2H), 7.76-7.75 (m, 2H), 7.38 (d, J=8.3 Hz, 2H), 2.45 (s, 3H).

¹³C NMR (150 MHz, CDCl₃): δ=166.8, 145.5, 134.2, 134.1, 130.7, 130.2, 129.9, 128.7, 128.0, 126.6, 125.1, 21.9.

HRMS (ESI-QTOF): m/z calculated for C₁₄H₁₃N₂O₄S⁺ [M+H]⁺: 305.0596; found: 305.0601.

### General Procedure (E) for the Synthesis of p-tolylsulfinimines 3n- and 3n

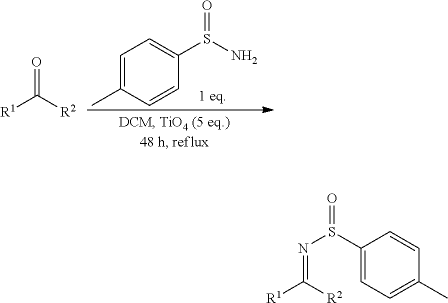

In a 100 mL two-necked flask fitted with a condenser, a septum, an argon inlet, and a magnetic bar, the carbonyl derivative (1 eq.) and racemic p-toluenesulfinamide (1 eq.) are dissolved in anhydrous dichloromethane (0.07 M). The reaction medium is stirred at ambient temperature then freshly distilled Ti(OEt)₄ (5 eq.) is added dropwise and the reaction is held stirred at reflux (60° C.) for 48 h. After total conversion (monitored by ¹H NMR), methanol and a few drops of NaHCO₃ are added until the titanium salts precipitate. The medium is then filtered through Na₂SO₄ and the residue is washed with ethyl acetate, then the solvent is evaporated under reduced pressure and the crude reaction is purified by silica gel chromatography to provide imine 3n or 3o.

### 4-methyl-N-(1-(4-nitrophenyl)ethylidene)benzenesulfinamide (3n)

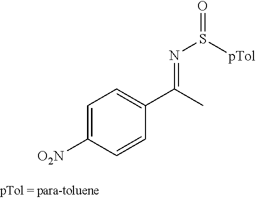

Prepared according to general procedure E by using 4-nitroacetophenone (500 mg; 3.03 mmol) then purified by silica gel chromatography (pentane/AcOEt 3/2; R_{f}=0.32) to provide 601 mg of imine 3n (66%) in the form of a yellow solid.

Melting point 103.7-105.7° C.

¹H NMR (600 MHz, CDCl₃): δ=8.25 (d, J=8.6 Hz, 2H), 8.01 (d, J=8.6 Hz, 2H), 7.73 (d, J=8.1 Hz, 2H), 7.35 (d, J=8.1 Hz, 2H), 2.84 (broad s, 3H), 2.42 (s, 3H).

¹³C NMR (150 MHz, CDCl₃): δ=149.7, 143.7, 142.7, 142.4, 130.2, 129.5, 128.6, 125.3, 123.8, 21.6, 20.4.

HRMS (ESI-QTOF): m/z calculated for C₁₅H₁₅N₂O₃S⁺[M+H]⁺: 303.0803; found: 303.0803.

### N-(1-(4-cyanophenyl)ethylidene)-4-methylbenzenesulfinamide (3o)

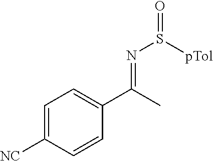

Prepared according to general procedure E by using 4-acetylbenzonitrile (500 mg; 3.44 mmol) then purified by silica gel chromatography (pentane/AcOEt 3/2; R_{f}=0.28) to provide 533 mg of imine 3o (55%) in the form of a yellow oil.

¹H NMR (600 MHz, CDCl₃): δ=7.95 (d, J=8.3 Hz, 2H), 7.72-7.68 (m, 4H), 7.34 (d, J=7.9 Hz, 2H), 2.80 (broad s, 3H), 2.41 (s, 3H).

¹³C NMR (150 MHz, CDCl₃): δ=171.6, 142.7, 142.4, 142.0, 132.4, 130.1, 128.1, 125.3, 118.2, 115.1, 21.6, 20.2.

HRMS (ESI-QTOF): m/z calculated for C₁₆H₁₅N₂OS⁺[M+H]⁺: 283.0905; found: 283.0902.

### General Procedure (F) for Synthesis of N-tosyl imines 3p and 3q by oxidation of N-tosylsulfinimines 3n and 3o, Respectively

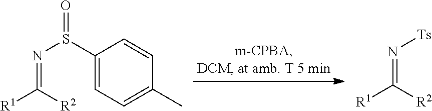

Dry mCPBA (1.1 eq.) is added at ambient temperature to a solution of p-tolylsulfinimine (1. eq.) in dichloromethane (0.2 M) then the reaction medium is held with magnetic stirring at ambient temperature for 5 minutes. After total conversion (monitored by 1H NMR), the solution is diluted with more dichloromethane and three washes in the presence of a saturated NaHCO₃ solution are performed. The solvent is then evaporated to provide imine 3p or 3q.

### 4-methyl-N-(1-(4-nitrophenyl)ethylidene)benzenesulfonamide (3p)

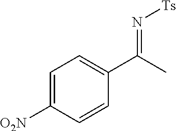 [SMILES: C/C(=N\S(=O)(=O)c1ccc(C)cc1)c1ccc([N+](=O)[O-])cc1]

Prepared according to general procedure F by using p-tolylsulfinimine 3n (121 mg; 0.4 mmol) to provide 127.3 mg of imine 3p (>99%) in the form of a yellow solid.

¹H NMR (600 MHz, CDCl₃): δ=8.25 (d, J=8.9 Hz, 2H), 8.03 (d, J=8.9 Hz, 2H), 7.92 (d, J=8.4 Hz, 2H), 7.38 (d, J=8.4 Hz, 2H), 3.03 (s, 3H), 2.47 (s, 3H).

¹³C NMR (150 MHz, CDCl₃): δ=150.4, 144.3, 143.1, 138.0, 130.2, 129.8, 129.4, 127.4, 123.9, 21.8, 21.6.

HRMS (ESI-QTOF): m/z calculated for C₁₅H₁₅N₂O₄S⁺[M+H]⁺: 319.0753; found: 319.0754.

### N-(1-(4-cyanophenyl)ethylidene)-4-methylbenzenesulfonamide (3q)

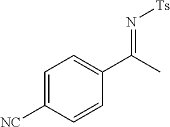 [SMILES: C/C(=N\S(=O)(=O)c1ccc(C)cc1)c1ccc(C#N)cc1]

Prepared according to general procedure F by using p-tolylsulfinimine 3o (120.4 mg; 0.43 mmol) to provide 128.3 mg of imine 3q (99%) in the form of a yellow solid.

Melting point 102.3-105.2° C.

¹H NMR (600 MHz, CDCl₃): δ=7.97 (d, J=8.6 Hz, 2H), 7.91 (d, J=8.3 Hz, 2H), 7.71 (d, J=8.6 Hz, 2H), 7.37 (d, J=8.3 Hz, 2H), 3.00 (s, 3H), 2.46 (s, 3H).

¹³C NMR (150 MHz, CDCl₃): δ=177.8, 144.3, 141.4, 138.1, 132.5, 129.8, 128.8, 127.4, 118.0, 116.3, 21.8, 21.4.

HRMS (ESI-QTOF): m/z calculated for C₁₆H₁₅N₂O₂S⁺[M+H]⁺: 299.0854; found: 299.0856.

### Synthesis of 1-(4-nitrophenyl)-N-(trimethylsilyl)methanimine (3r)

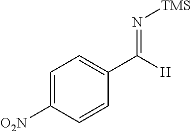 [SMILES: C[Si](C)(C)/N=C/c1ccc([N+](=O)[O-])cc1]

A solution of LiHMDS (3.7 mL, 3.65 mmol, 1.1 eq., 1 M in THF) is added dropwise to a solution of 4-nitrobenzaldehyde (500 mg, 3.31 mmol, 1 eq.) in anhydrous toluene (4 mL) at 0° C. The reaction mixture is then brought to ambient temperature and then held with stirring for one hour. When the reaction is finished (monitored by ¹H NMR), the solvent is evaporated to provide 735.8 mg of imine 3r (>99%) in the form of a brown oil.

¹H NMR (500 MHz, CDCl₃): δ=9.03 (s, 1H), 8.29 (d, J=8.8 Hz, 2H), 7.96 (d, J=8.8 Hz, 2H), 0.28 (s, 9H).

¹³C NMR (125 MHz, CDCl₃): δ=165.7, 149.5, 143.6, 129.2, 124.0, −1.2.

HRMS (ASAP-QTOF) m/z: not determined due to degradation of the product.

### General Procedure (G) for the Synthesis of diazirines 4 from N—R³ imines 3a to 3q

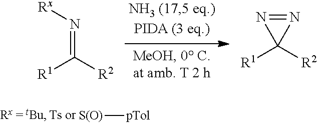

The ammonia solution (1.25 mL at 7 M in methanol; 17.5 eq.) is added into a Schlenk under argon containing N-tert-butyl imine (0.5 mmol; 1 eq.). The reaction mixture is cooled to 0° C. then the phenyliodonium diacetate obtained (1.5 mmol, 3 eq.) is added in a single portion. After 30 minutes at 0° C., the ice bath is removed and the reaction mixture is stirred for 1 h 30 min at ambient temperature. After total conversion, the medium is concentrated under reduced pressure then the crude reaction is purified by silica gel chromatography to provide diazirines 4a to 4g.

### 3-(4-nitrophenyl)-3H-diazirine (4a)

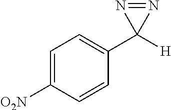 [SMILES: O=[N+]([O-])c1ccc(C2N=N2)cc1]

Prepared according to general procedure G by using imine 3a (103.2 mg; 0.5 mmol) or N-Ts imine 3h (152.2 mg, 0.5 mmol) then purified by silica gel chromatography (pentane/AcOEt 95/5; R_{f}=0.3) to provide 66 mg (80% from imine 3a) or 81.6 mg (>99% from N-Ts imine 3h) of diazirine 4a in the form of orange crystals.

¹H NMR (500 MHz, CDCl₃): δ=8.19 (d, J=8.0 Hz, 2H), 7.07 (d, J=8.0 Hz, 2H), 2.19 (s, 1H).

¹³C NMR (125 MHz, CDCl₃): δ=147.6; 143.6; 125.9; 123.6; 23.1.

### 3-(3-methyl-3H-diazirin-3-yl)propanoic acid (4b)

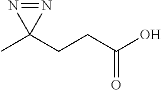

Prepared according to general procedure G by using imine 3b (85.6 mg; 0.5 mmol) then purified by silica gel chromatography (DCM/MeOH 95/5; R_{f}=0.3) to provide 64 mg of diazirine 4b (99%) in the form of a light yellow oil.

¹H NMR (500 MHz, CDCl₃): δ=2.24 (t, J=7.7 Hz, 2H), 1.72 (t, J=7.7 Hz, 2H), 1.05 (s, 3H).

¹³C NMR (125 MHz, CDCl₃): δ=178.0; 29.8; 29.3; 25.4; 19.8.

HRMS (ESI-QTOF): m/z calculated for C₅H₇N₂O₂⁻ [M]⁻: 127.0508; found: 127.0513.

### 4-(3H-diazirin-3-yl)benzonitrile (4c)

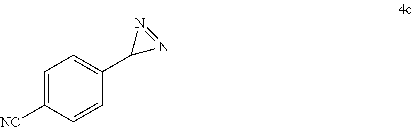 [SMILES: N#Cc1ccc(C2N=N2)cc1]

Prepared according to general procedure G by using imine 3c (93.2 mg; 0.5 mmol) or Ts imine 3i (142.2 mg, 0.5 mmol) then purified by silica gel chromatography (pentane/AcOEt 95/5; R_{f}=0.32) to provide 69.2 mg (97% from imine 3c) or 71.6 mg (>99% from Ts imine 3i) of diazirine 4c in the form of a yellow oil.

¹H NMR (600 MHz, CDCl₃): δ=7.61 (d, J=8.3 Hz, 2H), 7.01 (d, J=8.3 Hz, 2H), 2.13 (s, 1H).

¹³C NMR (150 MHz, CDCl₃): δ=141.8, 132.2, 125.9, 118.5, 111.9, 23.3.

### 1-(4-(3H-diazirin-3-yl)phenyl)ethan-1-one (4d)

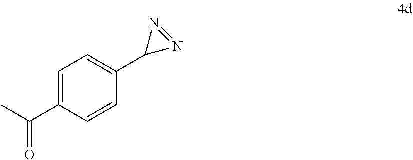

Prepared according to general procedure G by using either imine 3e (102.2 mg; 0.5 mmol) or Ts imine 3k (152.2 mg, 0.5 mmol) then purified by silica gel chromatography (pentane/AcOEt 95/5; R_{f}=0.14) to provide 39.4 mg (49% from imine 3e) or 55.7 mg (70% from Ts imine 3k) of diazirine 4d in the form of a yellow oil.

¹H NMR (600 MHz, CDCl₃): δ=7.91 (d, J=8.4 Hz, 2H), 7.00 (d, J=8.4 Hz, 2H), 2.59 (s, 3H), 2.13 (s, 1H).

¹³C NMR (150 MHz, CDCl₃): δ=197.5, 141.7, 136.7, 128.5, 125.5, 26.8, 23.4.

### Ethyl 4-(3H-diazirin-3-yl)benzoate (4e)

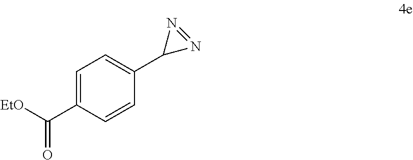 [SMILES: CCOC(=O)c1ccc(C2N=N2)cc1]

Prepared according to general procedure G by using either imine 3d (116.7 mg; 0.5 mmol) or Ts imine 3j (165.7 mg, 0.5 mmol) then purified by silica gel chromatography (pentane/AcOEt 95/5; R_{f}=0.5) to provide 57.8 mg (61% from imine 3d) or 89.1 mg (94% from Ts imine 3j) of diazirine 4e in the form of a yellow oil.

¹H NMR (500 MHz, CDCl₃): δ=7.99 (d, J=8.5 Hz, 2H), 6.97 (d, J=8.5 Hz, 2H), 4.37 (q, J=7.1 Hz, 2H), 2.11 (s, 1H), 1.39 (t, J=7.1 Hz, 3H).

¹³C NMR (125 MHz, CDCl₃): δ=166.2, 141.4, 130.3, 129.7, 125.2, 61.2, 23.5, 14.5.

### 3-(3-nitrophenyl)-3H-diazirine (4f)

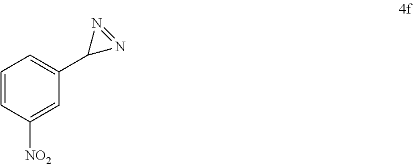 [SMILES: O=[N+]([O-])c1cccc(C2N=N2)c1]

Prepared according to general procedure G by using either imine 3f (103.2 mg; 0.5 mmol) or Ts imine 3l (152.2 mg, 0.5 mmol) then purified by silica gel chromatography (pentane/AcOEt 95/5; R_{f}=0.4) to provide 81.6 mg (>99% from imine 3f) or 81.6 mg (>99% from Ts imine 3l) of diazirine 4f in the form of a dark brown oil.

¹H NMR (600 MHz, CDCl₃): δ=8.18-8.16 (m, 1H), 7.82-7.81 (m, 1H), 7.51 (t, J=8.0 Hz, 1H), 7.24-7.23 (m, 1H), 2.20 (s, 1H).

¹³C NMR (150 MHz, CDCl₃): δ=148.4, 138.7, 131.0, 129.6, 123.1, 120.3, 23.0.

HRMS (ASAP-QTOF): m/z calculated for C₇H₆N₃O₂⁺ [M+H]⁺: 164.0460; found: 164.0462.

### 3-(2-nitrophenyl)-3H-diazirine (4g)

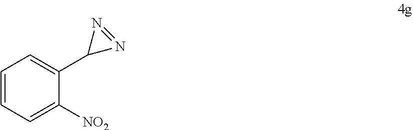 [SMILES: O=[N+]([O-])c1ccccc1C1N=N1]

Prepared according to general procedure G by using either imine 3g (103.2 mg; 0.5 mmol) or Ts imine 3m (152.2 mg, 0.5 mmol) then purified by silica gel chromatography (pentane/AcOEt 95/5; R_{f}=0.51) to provide 52.8 mg (65% from imine 3g) or 55.2 mg (68% from Ts imine 3m) of diazirine 4g in the form of a light yellow oil.

¹H NMR (600 MHz, CDCl₃): δ=8.11 (d, J=8.1 Hz, 1H), 7.54 (t, J=8.1 Hz, 1H), 7.45 (t, J=8.1 Hz, 1H), 6.50 (d, J=8.1 Hz, 1H), 2.94 (s, 1H).

¹³C NMR (150 MHz, CDCl₃): δ=149.8, 133.4, 132.2, 128.5, 125.9, 125.6, 19.9.

### 3-methyl-3-(4-nitrophenyl)-3H-diazirine (4h)

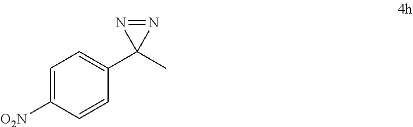

Prepared according to general procedure G by using either p-tolylsulfinimine 3n (151.2 mg; 0.5 mmol) or N-Ts imine 3p (127.3 mg, 0.4 mmol) then purified by silica gel chromatography (pentane/AcOEt 95/5; R_{f}=0.5) to provide 58.9 mg (67% from p-tolylsulfinimine 3n) or 64.1 mg (90% from N-Ts imine 3p) of diazirine 4h in the form of yellow crystals.

Melting point 52.3-55.5° C.

¹H NMR (600 MHz, CDCl₃): δ=8.20-8.18 (m, 2H), 7.08-7.06 (m, 2H), 1.59 (s, 3H).

¹³C NMR (150 MHz, CDCl₃): δ=147.15, 147.06, 126.4, 123.6, 26.5, 17.5.

### 4-(3-methyl-3H-diazirin-3-yl)benzonitrile (4i)

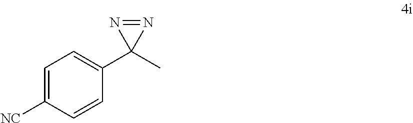

Prepared according to general procedure G by using either p-tolylsulfinimine 3o (141.2 mg; 0.5 mmol) or N-Ts imine 3q (90 mg, 0.3 mmol) then purified by silica gel chromatography (pentane/AcOEt 95/5; R_{f}=0.33) to provide 58.3 mg (74% from p-tolylsulfinimine 3o) or 43.5 mg (92% from N-Ts imine 3q) of diazirine 4i in the form of a yellow oil.

¹H NMR (600 MHz, CDCl₃): δ=7.62 (d, J=8.5 Hz, 2H), 7.01 (d, J=8.5 Hz, 2H), 1.55 (s, 3H).

¹³C NMR (150 MHz, CDCl₃): δ=145.1, 132.1, 126.3, 118.6, 111.3, 26.4, 17.4.

### General Procedure (H) for the Synthesis of ¹⁵N₂-diazirines 5 from N-tert-butyl imines

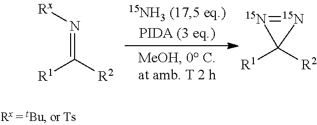

The ammonia solution labelled with nitrogen-15 (1.25 mL at 7 M in methanol; 17.5 eq.) is added into a Schlenk under argon containing N-tert-butyl imine or N-Ts imine (0.5 mmol; 1 eq.). The reaction mixture is cooled to 0° C. then the phenyliodonium diacetate obtained (1.5 mmol; 3 eq.) is added in a single portion. After 30 minutes at 0° C., the ice bath is removed and the reaction mixture is stirred for 1 h 30 min at ambient temperature. After total conversion, the medium is concentrated under reduced pressure then the crude reaction is purified by silica gel chromatography to provide diazirine 5.

### ¹⁵N₂-(3-(4-nitrophenyl)-3H-diazirine) (5a)

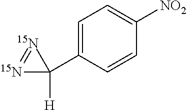 [SMILES: O=[N+]([O-])c1ccc(C2[15N]=[15N]2)cc1]

Prepared according to general procedure H by using imine 3a (103.2 mg; 0.5 mmol) or N-Ts imine 3h (152.2 mg, 0.5 mmol) then purified by silica gel chromatography (pentane/AcOEt 95/5; R_{f}=0.3) to provide 53 mg (64% from imine 3a) or 61.3 mg (74% from imine 3h) of diazirine 5a in the form of orange crystals.

¹H NMR (600 MHz, CDCl₃): δ=8.19 (d, J=7.5 Hz, 2H), 7.07 (d, J=7.5 Hz, 2H), 2.19 (t, J=2.2 Hz, 1H).

¹³C NMR (150 MHz, CDCl₃): δ=147.7; 143.7 (t, J=1.2 Hz), 126.0; 123.7; 23.2 (t, J=9.0 Hz).

¹⁵N NMR (60.8 MHz, CDCl₃): δ=422.0 (d, J=2.2 Hz).

¹⁵N{¹H} NMR (60.8 MHz, CDCl₃): δ=422.2 (broad s, 1N (¹⁵N=¹⁴N)-diazirine), 422.1 (broad s, 2N, ¹⁵N₂-diazirine)

¹⁵N₂-diazirine/(¹⁵N=¹⁴N)-diazirine ratio: >99% from N-tBu imine 3a; 71% from N-Ts imine 3h (according to ¹⁵N{¹H} NMR) measurements.

### ¹⁵N₂-(3-(3-methyl-3H-diazirin-3-yl)propanoic acid) (5b)

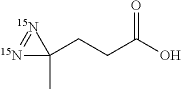

Prepared according to general procedure H by using imine 3b (85.6 mg; 0.5 mmol) then purified by silica gel chromatography (DCM/MeOH 95/5; R_{f}=0.3) to provide 22.4 mg of diazirine 5b (30%) in the form of a light yellow oil.

¹H NMR (600 MHz, CDCl₃): δ=9.52 (broad s, 1H), 2.23 (tt, J=7.8 and 2.2 Hz, 2H), 1.71 (t, J=7.8 Hz, 2H), 1.05 (t, J=0.8 Hz, 3H).

¹³C NMR (150 MHz, CDCl₃): δ=178.5; 29.5 (t, J=1.5 Hz, 1C); 28.6; 25.2 (t, J=10.5 Hz, 1C); 19.8 (t, J=1.5 Hz, 1C).

¹⁵N NMR (60.8 MHz, CDCl₃): δ=464.2.

¹⁵N{¹H} NMR (60.8 MHz, CDCl₃): δ=464.2 (broad s, 2N, ¹⁵N₂-diazirine).

¹⁵N₂-diazirine/(¹⁵N=¹⁴N)-diazirine ratio: >99% from N-tBu imine 3b (according to ¹⁵N{¹H} NMR) measurements.

### ¹⁵N₂-(3-(2-nitrophenyl)-3H-diazirine) (5c)

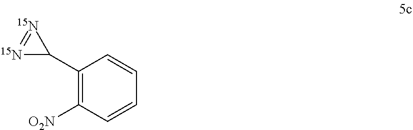 [SMILES: O=[N+]([O-])c1ccccc1C1[15N]=[15N]1]

Prepared according to general procedure H by using N-Ts imine 3m (152.2 mg; 0.5 mmol) then purified by silica gel chromatography (Pentane/AcOEt 95/5) to provide 8.4 mg of diazirine 5c (10%) in the form of a light yellow oil.

¹H NMR (600 MHz, CDCl₃): δ=8.11 (dd, J=7.9; 1.3 Hz, 1H), 7.54 (td, J=7.9; 1.3 Hz, 1H), 7.45 (m, 1H), 6.50 (dd, J=7.9; 1.3 Hz, 1H), 2.93 (t, J=2.4 Hz, 1H).

¹³C NMR (150 MHz, CDCl₃): δ=149.7, 133.3, 132.2 (t, J=1.3 Hz), 128.5, 125.9, 125.6, 19.8 (t, J=8.9 Hz).

¹⁵N NMR (60.8 MHz, CDCl₃): δ=422.7 (broad s, 2N, ¹⁵N₂-diazirine).

¹⁵N{¹H} NMR (60.8 MHz, CDCl₃): δ=422.8 (broad s, 1N (¹⁵N=¹⁴N)-diazirine), 422.7 (broad s, 2N, ¹⁵N₂-diazirine)

¹⁵N₂-diazirine/(¹⁵N=¹⁴N)-diazirine ratio: 84% from N-Ts imine 3m (according to ¹⁵N{¹H} NMR measurements).

### ¹⁵N₂-(3-(3-nitrophenyl)-3H-diazirine) (5d)

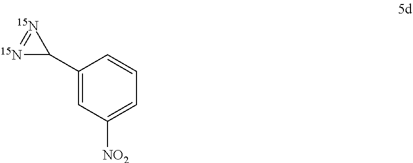 [SMILES: O=[N+]([O-])c1cccc(C2[15N]=[15N]2)c1]

Prepared according to general procedure H by using N-Ts imine 3l (152.2 mg; 0.5 mmol) then purified by silica gel chromatography (pentane/AcOEt 95/5) to provide 42.8 mg of diazirine 5d (52%) in the form of a dark brown oil.

¹H NMR (600 MHz, CDCl₃): δ=8.16 (ddd, J=8.0; 2.2; 1.0 Hz, 1H), 7.79 (t, J=2.2 Hz, 1H), 7.51 (t, J=8.0 Hz, 1H), 7.23 (ddd, J=8.0; 2.2; 1.0 Hz, 1H), 2.20 (t, J=2.4 Hz, 1H).

¹³C NMR (150 MHz, CDCl₃): δ=148.3, 138.6 (t, J=1.2 Hz), 131.0, 129.6, 123.0, 120.3, 22.9 (t, J=9.0 Hz).

¹⁵N NMR (60.8 MHz, CDCl₃): δ=423.9 (d, J=2.4 Hz, 2N, ¹⁵N₂-diazirine).

¹⁵N{¹H} NMR (60.8 MHz, CDCl₃): δ=424.0 (broad s, 1N (¹⁵N=¹⁴N)-diazirine), 423.9 (broad s, 2N, ¹⁵N₂-diazirine)

¹⁵N₂-diazirine/(¹⁵N=¹⁴N)-diazirine ratio: 53% from N-Ts imine 3l (according to ¹⁵N{¹H} NMR measurements).

### ¹⁵N₂-(4-(3H-diazirin-3-yl) ethyl benzoate) (5e)

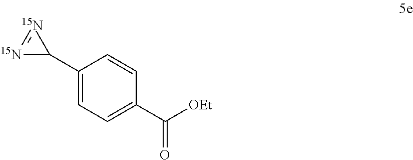 [SMILES: CCOC(=O)c1ccc(C2[15N]=[15N]2)cc1]

Prepared according to general procedure H by using N-Ts imine 3j (165.7 mg; 0.5 mmol) then purified by silica gel chromatography (pentane/AcOEt 95/5) to provide 20.4 mg of diazirine 5e (23%) in the form of a light yellow oil.

¹H NMR (600 MHz, CDCl₃): δ=7.99 (d, J=8.4 Hz, 2H), 6.96 (d, J=8.4 Hz, 2H), 4.37 (q, J=7.1 Hz, 2H), 2.11 (t, J=2.3 Hz, 1H), 1.38 (t, J=7.1 Hz, 3H).

¹³C NMR (150 MHz, CDCl₃): δ=166.2, 141.4 (t, J=1.1 Hz), 130.2, 129.6, 125.2, 61.2, 23.4 (t, J=9.0 Hz), 4.4.

¹⁵N NMR (60.8 MHz, CDCl₃): δ=424.3 (broad s, 2N, ¹⁵N₂-diazirine).

¹⁵N {¹H} NMR (60.8 MHz, CDCl₃): δ=424.4 (broad s, 1N (¹⁵N=¹⁴N)-diazirine), 424.3 (broad s, 2N, ¹⁵N₂-diazirine)

¹⁵N₂-diazirine/(¹⁵N=¹⁴N)-diazirine ratio: 77% from N-Ts imine 3j (according to ¹⁵N{¹H} NMR measurements).

### ¹⁵N₂-(1-(4-(3H-diazirin-3-yl)phenyl)ethanone) (5f)

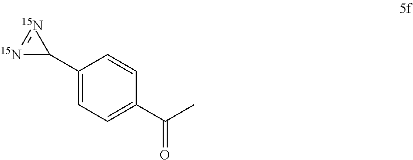

Prepared according to general procedure H by using N-Ts imine 3k (150.7 mg; 0.5 mmol) then purified by silica gel chromatography (pentane/AcOEt 95/5) to provide 20 mg of diazirine 5f (25% N-Ts imine 3k) in the form of a light yellow oil.

¹H NMR (600 MHz, CDCl₃): δ=7.90 (d, J=8.5 Hz, 2H), 6.99 (d, J=8.5 Hz, 2H), 2.59 (s, 3H), 2.12 (t, J=2.2 Hz, 1H).

¹³C NMR (150 MHz, CDCl₃): δ=197.5, 141.72 (t, J=1.2 Hz), 136.7, 128.5, 125.4, 26.8, 23.39 (t, J=9.0 Hz).

¹⁵N NMR (60.8 MHz, CDCl₃): δ=424.0 (d, J=2.5 Hz, 2N, ¹⁵N₂-diazirine).

¹⁵N{¹H} NMR (60.8 MHz, CDCl₃): δ=424.1 (broad s, 1N (¹⁵N=¹⁴N)-diazirine), 424.0 (broad s, 2N, ¹⁵N₂-diazirine)

¹⁵N₂-diazirine/(¹⁵N=¹⁴N)-diazirine ratio: 49% from N-Ts imine 3k (according to ¹⁵N{¹H} NMR measurements).

### ¹⁵N₂-(4-(3H-diazirin-3-yl)benzonitrile (5g)

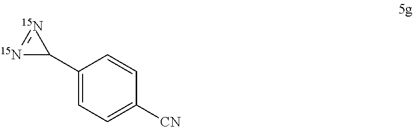 [SMILES: N#Cc1ccc(C2[15N]=[15N]2)cc1]

Prepared according to general procedure H by using N-Ts imine 3i (142.2 mg; 0.5 mmol) then purified by silica gel chromatography (pentane/AcOEt 95/5) to provide 20.4 mg of diazirine 5g (28%) in the form of a yellow oil.

¹H NMR (600 MHz, CDCl₃): δ=7.60 (d, J=8.6 Hz, 2H), 7.00 (d, J=8.6 Hz, 2H), 2.12 (t, J=2.4 Hz, 1H).

¹³C NMR (150 MHz, CDCl₃): δ=141.7 (t, J=1.2 Hz), 132.2, 125.9, 118.5, 111.8, 23.22 (t, J=9.0 Hz).

¹⁵N NMR (60.8 MHz, CDCl₃): δ=422.7 (d, J=2.4 Hz, 2N, ¹⁵N₂-diazirine).

¹⁵N{¹H} NMR (60.8 MHz, CDCl₃): δ=422.8 (broad s, 1N (¹⁵N=¹⁴N)-diazirine), 422.7 (broad s, 2N, ¹⁵N₂-diazirine)

HRMS (ASAP-QTOF): unstable product.

¹⁵N₂-diazirine/(¹⁵N=¹⁴N)-diazirine ratio: 92% from N-Ts imine 3i (according to ¹⁵N{¹H} NMR measurements).

Use of Diazirines According to the Invention in Photoaffinity Labelling

Diazirine 1a, derived from tyrosine, was solubilized in methanol then subjected to UV radiation (360 nm) for a duration of 16 h. The carbene generated in situ is then directly trapped by the reaction solvent, methanol, to lead to ether 6a (see figure below) with total conversion.

This reaction demonstrates that the diazirines of the present invention are usable in photoaffinity labelling.

Molecular probes are generally complex molecules, having particular groups such as fluorescent markers or groups having a particular affinity for their biological target. Thus, the inventors have shown that diazirines according to the invention can be easily functionalized. Diazirine 1a was therefore reacted with tert-butyl bromoacetate to lead to ester 7, then quickly hydrolyzed to obtain carboxylic acid 8.

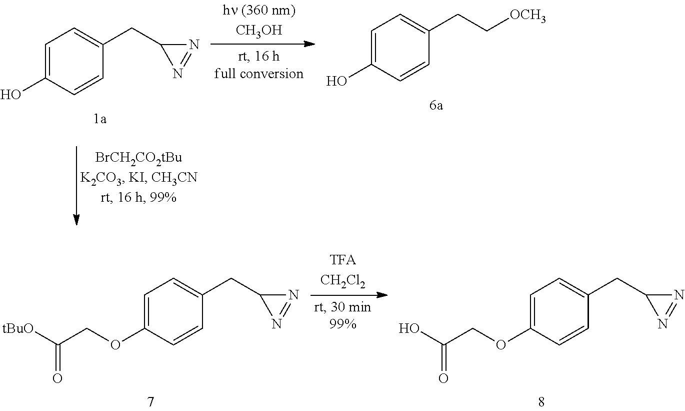 [SMILES: Oc1ccc(CC2N=N2)cc1 ; COCCc1ccc(O)cc1 ; CC(C)(C)OC(=O)COc1ccc(CC2N=N2)cc1 ; O=C(O)COc1ccc(CC2N=N2)cc1]

### Synthesis of 6a

0.4 mL of anhydrous methanol is added into a 5 mL single-necked flask, under argon, containing diazirine 1a (30 mg; 0.2 mmol). The solution is stirred with irradiation at 365 nm (Rayonet) for 16 h at ambient temperature. ¹H NMR confirms a total conversion, with the appearance of signals corresponding to ether 6a.

### Synthesis of 7

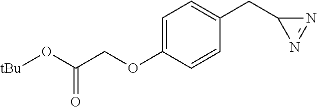 [SMILES: CC(C)(C)OC(=O)COc1ccc(CC2N=N2)cc1]

Potassium carbonate (0.75 mmol), potassium iodide (15 mol %) and then, dropwise, tert-butyl bromoacetate (0.625 mmol) are successively introduced into a solution of diazirine 1a (0.5 mmol) in acetonitrile (1.5 mL). The reaction medium is stirred for 24 h at ambient temperature, diluted with distilled water and then extracted with ethyl acetate. The organic phase is dried on magnesium sulfate and then filtered. After evaporation under vacuum, diazirine 7 is obtained (131 mg, 99%) in the form of an orange oil.

¹H NMR (500 MHz, CDCl₃): δ=7.18 (d, J=7.0 Hz, 2H), 6.86 (d, J=7.0 Hz, 2H), 4.51 (s, 2H), 2.49 (d, J=3.5 Hz, 2H), 1.49 (s, 9H), 1.09 (t, J=3.5 Hz, 1H).

¹³C NMR (125 MHz, CDCl₃): δ=168.2; 157.1; 130.1; 129.1; 115.0; 82.5; 65.9; 35.9; 28.2; 22.2.

HRMS (ESI⁺): m/z calculated for C₁₄H₁₈N₂O₃Na⁺ [M+Na]⁺: 285.1215; found: 285.1216.

### Synthesis of 8

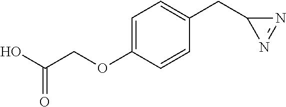 [SMILES: O=C(O)COc1ccc(CC2N=N2)cc1]

Trifluoroacetic acid (1 mL) is added dropwise to a solution of diazirine 7 (0.4 mmol) in dichloromethane (1 mL). After magnetic stirring of the reaction medium for 1 h at ambient temperature, it is evaporated and then the residue is dissolved in diethyl ether. The organic phase is dried on magnesium sulfate and then filtered. After evaporation, diazirine 8 (82 mg; 99%) is obtained in the form of a beige solid.

¹H NMR (500 MHz, CDCl₃): δ=7.20 (d, J=8.7 Hz, 2H), 6.90 (d, J=8.7 Hz, 2H), 4.68 (s, 2H), 2.51 (d, J=4.4 Hz, 2H), 1.10 (t, J=4.4 Hz, 1H).

¹³C NMR (125 MHz, CDCl₃): δ=173.4; 156.5; 130.3; 129.8; 115.1; 65.0; 35.8; 22.1.

HRMS (ESI⁻): m/z calculated for C₂₀H₉N₂O₃⁻ [M−H]⁻: 205.0613; found: 205.0603.

1. One-pot synthesis method for a diazirine, wherein the nitrogen atoms each correspond, independently of one another, to the ¹⁴N isotope or the ¹⁵N isotope, from an amino acid or an imine comprising the reaction of the amino acid or imine with ammonia of formula ¹⁴NH₃ or ¹⁵NH₃ and an oxidant containing a hypervalent iodine atom.

2. Method according to claim 1, wherein the diazirine corresponds to formula (I) below: 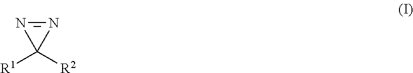
or a physiologically acceptable salt or solvate thereof, a stereoisomer or a mixture of stereoisomers in any proportions,
wherein the nitrogen atoms each correspond, independently of one another, to the ¹⁴N isotope or the ¹⁵N isotope,
wherein R¹ represents H, V, W or V—W where:
V represents an aliphatic chain where up to 8 methylene units of the aliphatic chain are optionally replaced by O, C(O), S, S(O), S(O)₂, NR′ or SiR′R″; V is optionally substituted by 1 to 12 groups selected from OH, NR′R″, halogen, CN, oxo, (═NR′) or aryl,
W represents a cycloalkyl, an aryl, a heterocycle or a heteroaryl; W is optionally substituted by 1 to 4 groups selected from halogen, CN, NO₂, OH, NR′R″ an aliphatic chain, where up to 4 methylene units of the aliphatic chain are optionally replaced by O, C(O), S, S(O), S(O)₂, NR′ or SiR′R″,
R′ and R″ each represent, independently of one another, H or an aliphatic chain, where up to 4 methylene units of the aliphatic chain are optionally replaced by O, C(O), S, S(O), S(O)₂, NR′ or SiR′R″, and
R² represents H or a (C₁-C₁₀) aliphatic chain, where up to 4 methylene units of the aliphatic chain are optionally replaced by O, C(O), S, S(O), S(O)₂, NR′ or SiR′R″,
said method comprising the following steps:
(a) Reaction of a compound of formula (II) below: 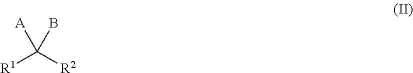
or a physiologically acceptable salt or solvate thereof, a stereoisomer or a mixture of stereoisomers in any proportions,
wherein A represents NH₂, the nitrogen atom being the ¹⁴N isotope or the ¹⁵N isotope, and B represents COOH and R¹ and R² are as defined above,
or A-B forms a single imine group of formula ═NR³, and R³ represents V, W or V—W as defined above,
with an oxidant comprising a hypervalent iodine atom of formula (III) below: 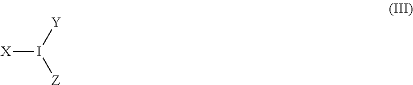
wherein X represents a (C₁-C₁₀) alkyl, an aryl or a heteroaryl; X being optionally substituted with 1 to 12 groups selected from OH, NR′R″, halogen, CN, oxo, (═NR′) or aryl,
Y and Z represent, independently of one another, OH, a halogen, NH₂, CN, a (C₁-C₁₀)alkyl group, ((C₁-C₁₀)alkyl)-aryl group or a ((C₁-C₁₀)alkyl)-heteroaryl group, where up to 4 methylene units of said alkyl are optionally replaced by O, C(O), S(O)₂ or NH,
and in the presence of ¹⁴NH₃ or ¹⁵NH₃ ammonia, in a reaction solvent
(b) Purification of the diazirine of formula (I) obtained in step (a).

3. Method according to claim 1, wherein the ammonia source is pure gaseous or liquid ammonia or ammonia in solution in a solvent.

4. Method according to claim 2, wherein the compound of formula (II) is an amino acid of formula (IIa) below: 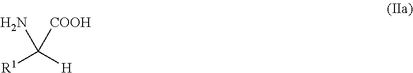
or a physiologically acceptable salt or solvate thereof, a stereoisomer or a mixture of stereoisomers in any proportions,
wherein the nitrogen atom of the amine function bound to the carbon also bearing the carboxylic acid function COOH, is either the ¹⁴N isotope or the ¹⁵N isotope, and
R¹ is as defined in claim 2.

5. Method according to claim 4, wherein it comprises a preliminary protection step of compound (IIa).

6. Method according to claim 4, wherein compound (IIa) is one of the following amino acids: L-aspartic acid, L-asparagine, L-glutamine, L-glycine, L-alanine, L-valine, L-isoleucine, L-leucine, L-phenylalanine, L-tryptophan, 4-methyl-L-phenylalanine, L-Histidine, L-tyrosine, L-glutamic acid and the following other protected amino acids: N_{ε}-acetyl-L-lysine, N_{ε}-benzyloxycarbamate-L-lysine, L-methionine sulfoxide, S-trityl-L-cysteine, S-benzyl-L-cysteine, O-benzyl-DL-serine, O-tert-butyldiphenylsilyl-DL-serine, O-tert-butyldiphenylsilyl-DL-threonine,1-(tert-butoxycarbonyl)-L-tryptophan,
4-iodophenyl-alanine, L-theanine, γ-benzyl-L-glutamic acid.

7. Method according to claim 2, wherein the compound of formula (II) is an imine of formula (IIb) below: 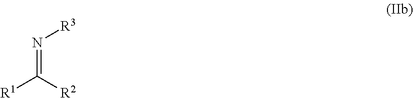
or a physiologically acceptable salt or solvate thereof, a stereoisomer or a mixture of stereoisomers in any proportions,
wherein R¹, R² and R³ are as defined above.

8. Method according to claim 7, wherein the imine of formula (IIb) is obtained from a carbonyl compound of formula (IV) below: 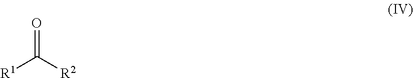
or a physiologically acceptable salt or solvate thereof, a stereoisomer or a mixture of stereoisomers in any proportions,
wherein R¹ and R² are as defined in claim 2.
comprising the reaction of the carbonyl compound of formula (IV) with an amine of formula (V) below:
R³—NH₂ (V)
wherein R³ is as defined in claim 2.

9. Method according to claim 1, wherein when the ammonia used is enriched in nitrogen-15, of formula ¹⁵NH₃, it is obtained from the method comprising the following steps:
(a′) reaction of ammonium chloride enriched in nitrogen-15 of formula ¹⁵NH₄Cl with
an alcoholate of formula R^{x}O-L in the corresponding anhydrous alcohol R^{x}OH, where
R^{x} is an alkyl group, and
L is a counterion chosen from Na⁺, K⁺ and Li⁺, preferably Na⁺,
(b′) optionally, elimination of the L-Cl salt formed,
the ammonium chloride enriched in nitrogen-15 and the alcoholate of formula R^{x}O-L being used in equivalent amounts.

10. Diazirines derived from amino acids obtainable by the method according to claim 4 corresponding to formula (I-A) below: 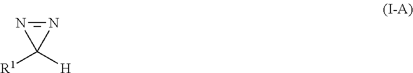
wherein R¹ is as defined in claim 1.

11. Diazirines obtainable by the method according to claim 1 corresponding to the following formulas:

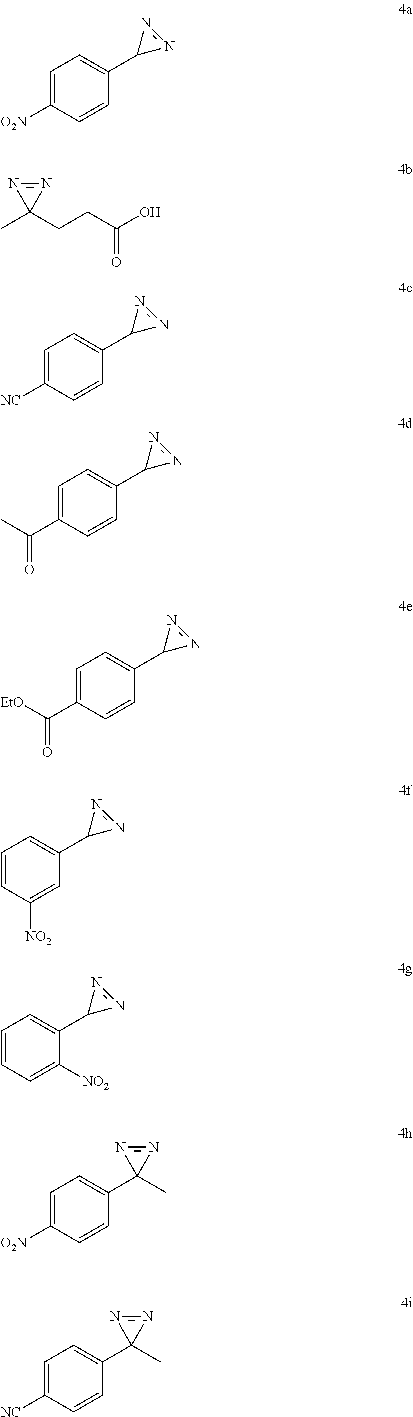

12. Diazirines obtainable by the method according to claim 1 corresponding to formula (I′) below: 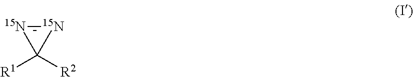
or a physiologically acceptable salt thereof, a stereoisomer or a mixture of stereoisomers in any proportions, wherein:
R¹ represents H, V, W or V—W where:
V represents an aliphatic chain where up to 4 methylene units of the aliphatic chain are optionally replaced by O, C(O), S, S(O), S(O)₂, NR′ or SiR′R″; V is optionally substituted by 1 to 12 groups selected from OH, NR′R″, halogen, CN, oxo, (═NR′) or aryl,
W represents a cycloalkyl, an aryl, a heterocycle or a heteroaryl; W is optionally substituted by 1 to 4 groups selected from halogen, CN, NO₂, OH, NR′R″ an aliphatic chain, where up to 4 methylene units of the aliphatic chain are optionally replaced by O, C(O), S, S(O), S(O)₂, NR′ or SiR′R″,
R′ and R″ each represent, independently of one another, H or an aliphatic chain, where up to 4 methylene units of the aliphatic chain are optionally replaced by O, C(O), S, S(O), S(O)₂, NR′ or SiR′R″, and
R² represents H or an aliphatic chain, where up to 4 methylene units of the aliphatic chain are optionally replaced by O, C(O), S, S(O), S(O)₂, NR′ or SiR′R″,
with the condition that, when R² is a methyl group, R¹ is preferably a phenyl substituted by a CN, NO₂, C(O)—(C₁-C₄ alkyl) or C(O)O—(C₁-C₄ alkyl) group.

13. Diazirines according to claim 12, wherein they correspond to formula (I′-A) below: 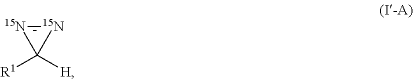
wherein R¹ is as defined in claim 12.

14. A method for photoaffinity labelling comprising using the diazirines according to claim 10.

15. A method for hyperpolarization comprising using the diazirines according to claim 12.

16. Method according to claim 2, wherein the oxidant is phenyliodonium diacetate (PIDA).

17. Method according to claim 2, wherein step (a) is carried out at a temperature comprised between 0° C. and ambient temperature and the reaction solvent is an alcohol.

18. method according to claim 9, wherein said method takes place at a temperature comprised between 0° C. and ambient temperature.

19. Diazirines derived from amino acids according to claim 10, wherein the diazirines conform to the following formulas:

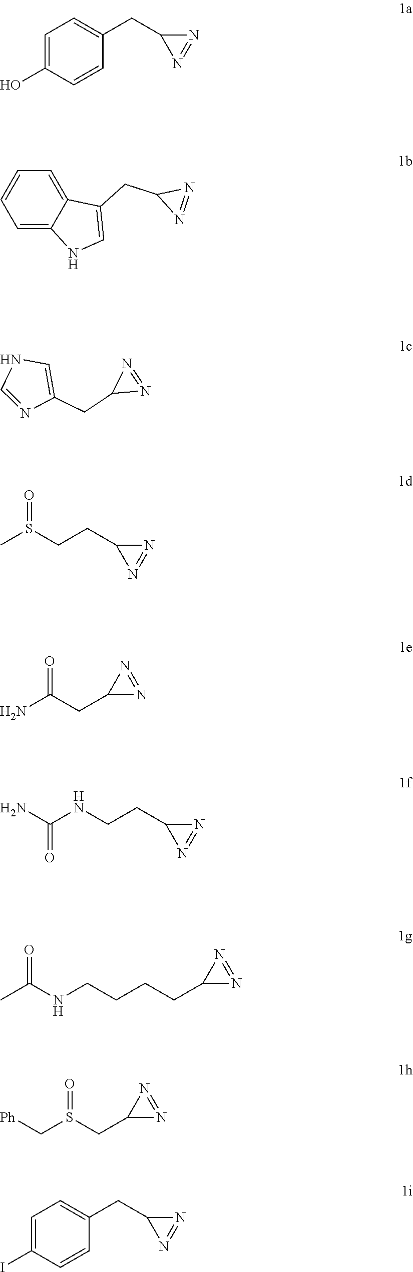

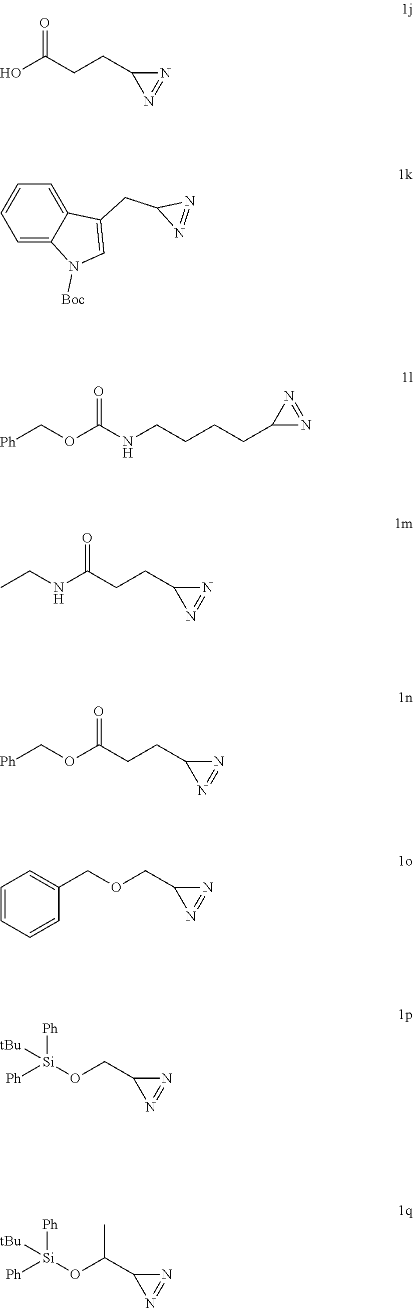

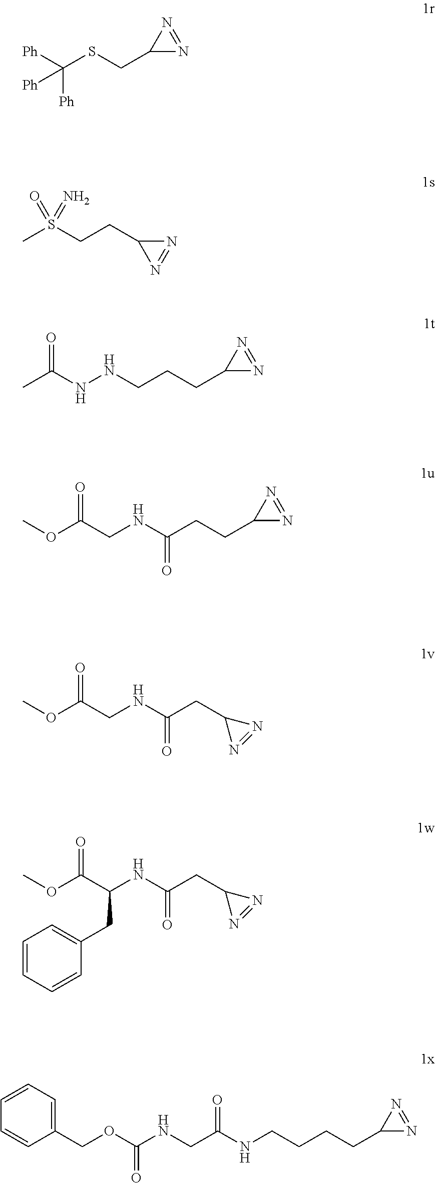

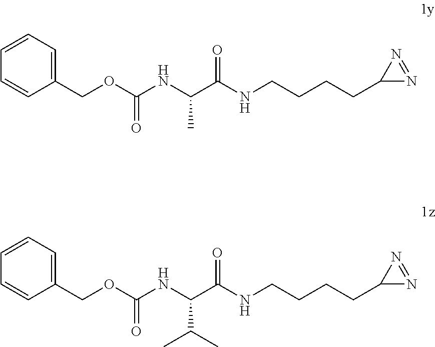 [SMILES: C[C@H](NC(=O)OCc1ccccc1)C(=O)NCCCCC1N=N1 ; CC(C)[C@H](NC(=O)OCc1ccccc1)C(=O)NCCCCC1N=N1]

20. Diazirines according to claim 12, wherein the diazirines of formula (I′) correspond to the following formulas: 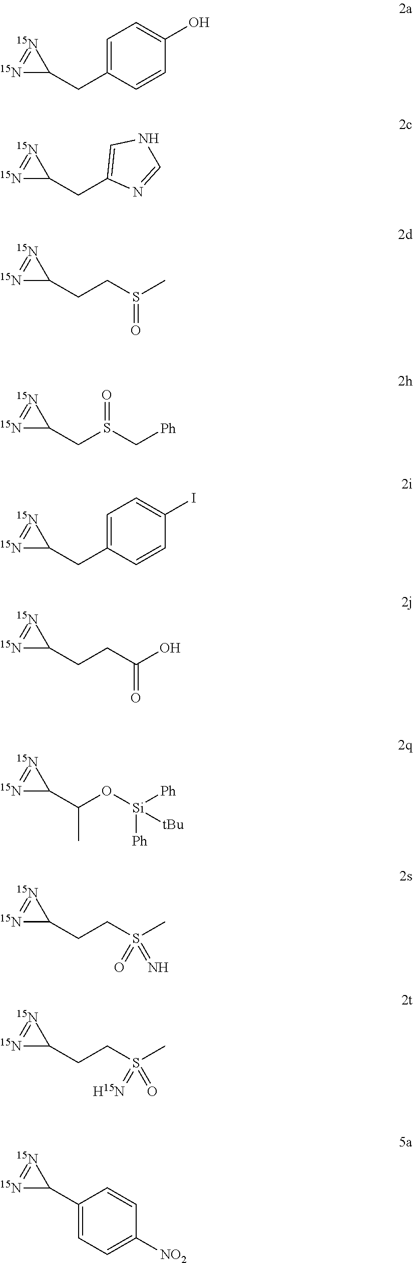 [chemical structure]
